(19) United States
(12) Patent Application Publication
(10) Pub. No.: US 2020/0334921 A1
(43) Pub. Date: Oct. 22, 2020

(54) METHODS FOR PROCESS VALIDATION

(71) Applicant: APOTEX INC., Toronto (CA)

(72) Inventors: Jana SPES, Toronto (CA); Ajay PAZHAYATTIL, Toronto (CA); Shu CHEN, Toronto (CA); Daniel ALSMEYER, San Mateo, CA (US)

(21) Appl. No.: 16/304,994

(22) PCT Filed: May 26, 2017
(86) PCT No.: PCT/IB2017/053134

§ 371 Date: Nov. 27, 2018

Related U.S. Application Data
(60) Provisional application No. 62/342,846, filed on May 27, 2016.
(60) Provisional application No. 62/342,852, filed on May 27, 2016.
(60) Provisional application No. 62/342,858, filed on May 27, 2016.

(51) Int. Cl. G07C 3/14; G06Q 10/06; G06Q 50/04; G06F 17/18; G16H 20/10

(52) U.S. Cl. CPC G07C 3/14; G06Q 10/06395; G16H 20/10; G06F 17/18; G06Q 50/04

(57) ABSTRACT

The present description relates to methods for physical process validation, qualification or verification. The methods are useful in a product lifecycle approach for the manufacture of physical products.

This application claims the benefit of priority of U.S. Provisional Application No. 62/342,846, filed on May 27, 2016, and of U.S. Provisional Application No. 62/342,852, filed on May 27, 2016, and of U.S. Provisional Application No. 62/342,858, filed on May 27, 2016, The content of each of the foregoing applications is incorporated herein in its entirety by reference.

### FIELD

The present description relates to methods for process validation, qualification or verification. The methods are useful in a product lifecycle approach for the manufacture of pharmaceuticals, biopharmaceuticals, nutraceuticals, cosmetics, food products, medical devices or other related products.

### BACKGROUND

A product lifecycle approach was introduced by the International Conference on Harmonization of Technical Requirements for Registration of Pharmaceuticals for Human Use (ICH) and adopted by the United States Food and Drug Administration in 2011 [FDA Guidance for Industry. Process Validation: General Principles and Practices, published by the U.S. Department of Health and Human Services, Food and Drug Administration in January 2011, incorporated herein in its entirety by reference]. This guidance outlines the general principles and approaches that the FDA considers appropriate elements of process validation for the manufacture of human and animal drug and biological products, including active pharmaceutical ingredients. In this guidance, process validation is defined as the “collection and evaluation of data, from the process design stage throughout commercial production, which establishes scientific evidence that a process is capable of consistently delivering quality products. Process validation involves a series of activities over the lifecycle of the product and process.”

The three stages of process validation in the FDA lifecycle approach include Stage 1—Process Design, Stage 2—Process Qualification, and Stage 3—Continued Process Manufacturing, as illustrated in FIG. 1. A primary goal of Stage 1 is to design a process suitable for routine commercial manufacturing that can consistently deliver a quality product. In Stage 2, the process design is evaluated to determine if the process is capable of reproducible commercial manufacture. In Stage 3, the process is monitored and maintained to assure that the process remains in a state of control during commercial manufacture.

### SUMMARY

The new process validation guidance included significant changes as compared to the 1987 guidance. The 2011 FDA Process Validation Guidance adds emphasis to process design, includes discussion of risk, involves activities over the entire process lifecycle in three defined stages, emphasizes the role of objective measures and statistical tools, and emphasizes knowledge, detection, and control of variability. Although it provides a framework to create a scientific approach to demonstrate process predictability, it presents some new challenges to industry. It leads to the development of new science and risk based decision making methodologies including, but not limited to, a scientific method to determine the number of qualification batches, new indicators of drug and process robustness, and fit for purpose risk assessment tools. Thus, novel tools are desired to facilitate data driven conclusions for the purpose of meeting regulatory guidance and product approval submission requirements.

From a regulatory perspective, another consideration with respect to process validation is the very nature of the manufacturing process. Although pharmaceutical products have historically been manufactured by a “batch” process, there is nothing in FDA regulations that prohibits their manufacture by a “continuous” process, Indeed, continuous manufacturing (CM) has the potential to lower capital costs, reduce inventory, and increase process efficiency among other benefits. But, the use of continuous processes involves the implementation of multivariate analysis for determination of product quality.

Accordingly, there is a need to develop new methods that apply scientific knowledge, risk analysis, experimental data, and continued process monitoring through the three phases of the process validation lifecycle. Further, the new methods should be consistent for various types of processes that include, for example, batch manufacturing, continuous manufacturing, and hybrid processes that include both batch and continuous manufacturing.

For example, one difficulty in a product lifecycle approach is performing a scientific, knowledge based, data driven technical risk assessment using objective measures. It would be desirable to understand the impact of process parameters and material attributes on quality attributes that impact the production operation.

According to an aspect, there is provided a method for analysis of a product manufacturing process, the method comprising: identifying a critical quality attribute (CQA) of a product manufactured by the process; identifying a plurality of material attributes, a plurality of processing parameters, or both, for the CQA, wherein each material attribute and/or processing parameter has a risk attribute for the critical quality attribute based on data; classifying each of the material attributes and/or processing parameters, based on the associated risk attribute of the material attribute and/or processing parameter, for the CQA into a category of a plurality of categories, each category having an assigned risk; and determining, by a hardware computer processor, a risk ratio for the critical quality attribute (CQA) based on the classification of the material attributes and/or process parameters into the categories.

According to an aspect, there is provided a computer program product comprising a non-transitory computer readable storage medium comprising computer-readable program code embodied therewith, the computer readable program code comprising computer readable program code configured to perform the method for analysis of a product manufacturing process.

Further still, there is provided a system to analyze a product manufacturing process, the system comprising: a hardware computer processor; and computer readable program code, when processed by the processor, configured to: enable identification of a critical quality attribute (CQA) of a product manufactured by the process; enable identification of a plurality of material attributes, a plurality of processing parameters of the process, or both, for the CQA, wherein each material attribute and/or processing parameter has a risk attribute for the critical quality attribute based on data; classify each of the product attributes and/or processing parameters, based on the associated risk attribute of that material attribute and/or processing parameter, for the CQA into a category of a plurality of categories, each category having an assigned risk; and determine a risk ratio for the critical quality attribute (CQA) based on the classification of the material attributes and/or process parameters into the categories.

In a further aspect, the 2011 FDA Process Validation Guidance indicates that state of control is a condition in which the set of controls consistently provides assurance of continued process performance and product quality. So, process validation is a collection and evaluation of data, from the process design stage through commercial production, which establishes scientific evidence that a process is capable of consistently delivering quality products. And, process qualification is confirmation that the manufacturing process as designed is capable of reproducible commercial manufacture. Thus, statistically sound methods are desired to scientifically determine the number of lots needed for process qualification and/or verification.

According to an aspect, there is provided a method for determining a minimum number of lots needed for performance qualification and/or verification of a product manufacturing process, the method comprising: identifying a critical quality attribute (CQA) of a product manufactured by the process; identifying a specification limit for the CQA; based on data collected for the CQA of a plurality of units of the product manufactured by the process, computing, by a hardware computer processor, one or more confidence bands for a plurality of statistical measures of the data collected; and determining a minimum number of lots required for the confidence band to fall within the specification limit for the CQA.

According to an aspect, there is provided a computer program product comprising a non-transitory computer readable storage medium comprising computer-readable program code embodied therewith, the computer readable program code comprising computer readable program code configured to perform the method for determining a minimum number of lots needed for performance qualification and/or verification of a product manufacturing process.

Further still, a system is provided to determine a minimum number of lots needed for performance qualification and/or verification of a product manufacturing process, the system comprising: a hardware computer processor; and computer readable program code, when processed by the processor, configured to: enable identification of a critical quality attribute (CQA) of a product manufactured by the process; enable identification of a specification limit for the CQA; based on data collected for the CQA of a plurality of units of the product manufactured by the process, compute, based on a number of lots, one or more confidence bands for a plurality of statistical measures of the data collected; and determine a minimum number of lots required for the confidence band to fall within the specification limit for the CQA.

In a further aspect, the acceptance criteria for pharmaceutical products are often multi-level, and traditional capability measures fall short in providing a suitable assessment of product risk. Thus, an applicable risk-based predictive method is desired to provide a reliable understanding of the ability of a product to fulfill acceptance requirements for quality attributes measuring product/process robustness.

According to an aspect, there is provided a method for risk assessment in a product manufacturing process, the method comprising: identifying a critical quality attribute (CQA) of a product manufactured by the process; identifying an acceptance criterion for the CQA for single and/or multiple units of the product manufactured by the process; based on data collected for the CQA of a plurality of units of the product manufactured by the process, determining, by a hardware computer processor, a Probability of Acceptance (Pa) value that specifies the probability that the acceptance criterion for a future unit of the product will meet or cross, or will not meet or not cross, a threshold defined for the acceptance criterion.

According to an aspect, there is provided a computer program product comprising a non-transitory computer readable storage medium comprising computer-readable program code embodied therewith, the computer readable program code comprising computer readable program code configured to perform the method for risk assessment in a product manufacturing process.

According to an aspect, there is provided a system to assess risk in a product manufacturing process, the system comprising: a hardware computer processor; and computer readable program code, when processed by the processor, configured to: enable identification of a critical quality attribute (CQA) of a product manufactured by the process; enable identification of an acceptance criterion for the CQA for single and/or multiple units of the product manufactured by the process; and based on data collected for the CQA of a plurality of units of the product manufactured by the process, determine a Probability of Acceptance (Pa) value that specifies the probability that the acceptance criterion for a future unit of the product will meet or cross, or will not meet or not cross, a threshold defined for the acceptance criterion.

Other aspects and features will be in part apparent and in part pointed out hereinafter.

### BRIEF DESCRIPTION OF THE DRAWINGS

Embodiments will now be described, by way of example only, with reference to the accompanying schematic drawings in which corresponding reference symbols indicate corresponding parts.

FIG. 1 depicts Stages 1-3 of the 2011 FDA Process Validation Guidance;

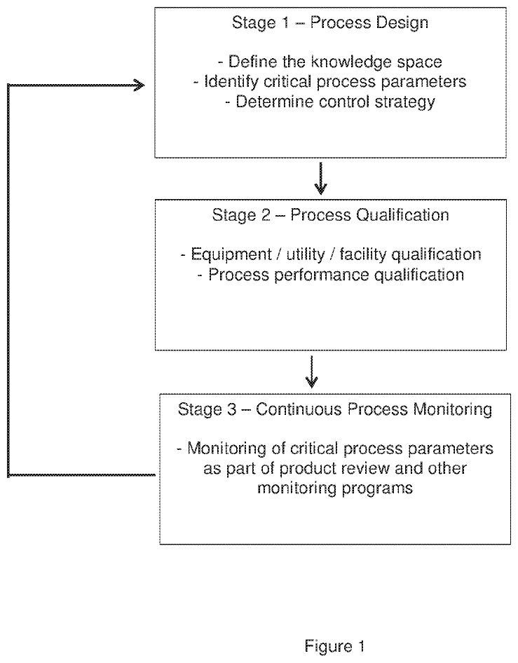

FIG. 2 depicts an example flowchart for a process validation lifecycle approach;

FIG. 3 depicts an example flowchart for a technical risk assessment method;

FIG. 4 depicts the relationship between CMAs, CPPs and CQAs;

FIG. 5A depicts a decision tree for a non-label claim CQA;

FIG. 5B depicts a decision tree for a label claim CQA;

FIG. 6 depicts a heat map for scale-up risk;

FIG. 7 depicts a flowchart for a method to determine a number of performance qualification lots;

FIG. 8 depicts a number of PPQ batches for an assay example;

FIG. 9 depicts a number of PPQ batches for a content uniformity example;

FIG. 10 depicts number of PPQ batches for a dissolution example;

FIG. 11A depicts an example batch-to-batch standard deviation for content uniformity;

FIG. 11B depicts an example batch-to-batch standard deviation for dissolution;

FIG. 12A depicts an example of batch-to-batch variability of content uniformity as a function of product strength;

FIG. 12B depicts an example of batch-to-batch variability of dissolution as a function of product strength;

FIG. 13 depicts a flowchart for a Probability of Acceptance analysis;

FIG. 14 depicts a flowchart for a Monte Carlo simulation for immediate release dissolution testing;

FIG. 15A depicts an example Probability of Acceptance chart for immediate release dissolution tests at Stage 1;

FIG. 15B depicts an example Probability of Acceptance chart for immediate release dissolution tests at Stage 2;

FIG. 15C depicts an example Probability of Acceptance chart for immediate release dissolution tests at Stage 3;

FIG. 16 depicts an example flow chart describing a Monte Carlo simulation process for dissolution; and

FIG. 17 depicts a block diagram that illustrates a computer system.

### DETAILED DESCRIPTION

To meet the 2011 FDA Process Validation Guidance for process validation, it is desirable to understand the sources of variation in a process, detect and measure sources of variation, understand the impact of variation on the process and final product attributes, and control the sources of variation commensurate with the risk they represent to the process and final product attributes. Process validation is applicable to a wide range of products, including but not limited to, an active pharmaceutical ingredient, a human drug product, a biological product, a radiopharmaceutical, a veterinary drug product, a dietary supplement, a nutraceutical, a cosmetic, and/or an excipient.

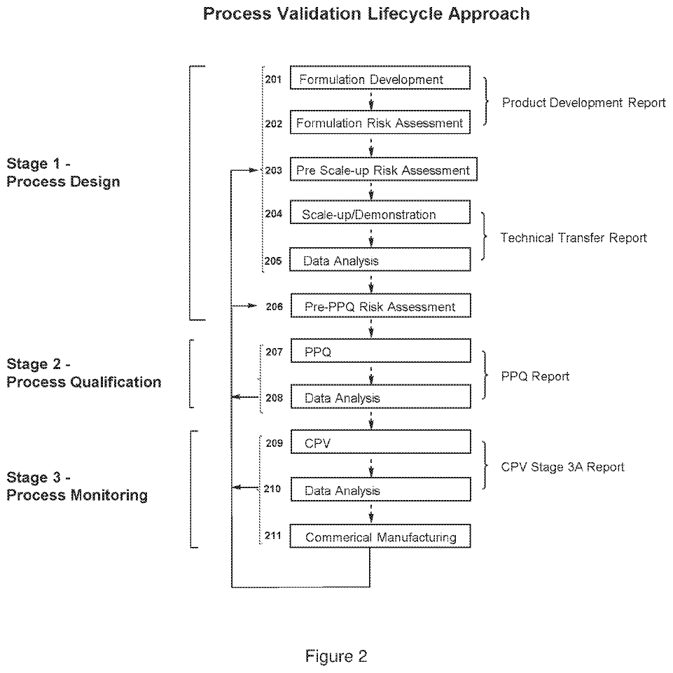

FIG. 2 is a flowchart for an example Process Validation Lifecycle Approach, showing the steps in Stage 1 (Process Design), Stage 2 (Process Qualification) and Stage 3 (Process Monitoring). The steps in Stage 1 include formulation development (201), formulation risk assessment (202), pre scale-up risk assessment (203), scale-up/demonstration of manufacturing (204), data analysis (205) and pre process performance qualification (PPQ) risk assessment (206). The steps in Stage 2 include process performance qualification (PPQ) (207) and data analysis (208). The steps in Stage 3 include continued process verification (CPV) (209), data analysis (210) and commercial manufacturing (211). As shown, results and data from various parts of the steps can flow to other steps.

The term “acceptance criteria” as used herein means the product specifications or acceptance/rejection criteria, such as acceptable quality level or unacceptable quality level. A sampling plan would typically be associated therewith to enable making of a decision whether to accept or reject, e.g., a lot or batch (or any other convenient subgroup of manufactured units).

The term “active ingredient” as used herein means any component that is intended to furnish pharmacological activity or other direct effect in the diagnosis, cure, mitigation, treatment, or prevention of disease, or to affect the structure or any function of the body of man or other animals. The term includes those components that may undergo chemical change in the manufacture of the drug product and be present in the drug product in a modified form intended to furnish the specified activity or effect.

The term “batch” as used herein means a specific quantity of a product that is intended to have uniform character and quality, within specified limits, and is produced according to a single manufacturing order during a same cycle of manufacture. Batch refers to the quantity of material and does not specify a mode of manufacture.

The term “batch process” as used herein means a process wherein all materials are charged before the start of processing and all products are discharged at the end of processing. There are two classes of “semi-batch” processes. In a fed-batch process, some or all materials are fed continuously during the process (or during some time of the processing). When processing is finished the products are removed batchwise. Examples include a wet granulation manufacturing process or a fermentation manufacturing process. In a batch-product removal process, materials are fed to the process before processing begins and the products (or some of the products) are removed continuously as processing occurs. Examples include a roller compaction manufacturing process or a tablet compression manufacturing process.

The term “biological product” as used herein means a virus, therapeutic serum, toxin, antitoxin, vaccine, blood, blood component or derivative, allergenic product, protein (except any chemically synthesized polypeptide), or analogous product, or arsphenamine or derivative of arsphenamine (or any other trivalent organic arsenic compound) applicable to the prevention, treatment or cure of a disease or condition of human beings or other animals. In contrast to chemically synthesized small molecular weight drugs, which have a well-defined structure and can be thoroughly characterized, biological products are generally derived from living material (e.g., human, animal, or microorganism), are complex in structure, and thus are usually not fully characterized. Biological products can be composed of sugars, proteins, or nucleic acids, or a combination of these substances. They may also be living entities, such as cells and tissues.

The term “component” as used herein means any ingredient intended for use in the manufacture of a product (such as a drug product), including those that may not appear in such product.

The term “continuous process” as used herein means a process composed of integrated (physically connected) continuous unit operations with zero or minimal hold volume in between. Examples include a hot melt extrusion manufacturing process, petroleum refining process, or many food processing processes. A “semi-continuous” process is a continuous process operated for a discrete time period. Examples include a milling manufacturing process, a roller compaction manufacturing process, or a tablet compression manufacturing process.

The term “critical material attribute” or “CMA” as used herein means a physical, chemical, biological or microbiological property or characteristic of an input material that should be within an appropriate limit, range, or distribution to ensure the desired quality of the output product.

The term “critical process parameter” or “CPP” as used herein means a process parameter whose variability has an impact on a critical quality attribute and therefore should be monitored or controlled to ensure the process produces the desired product.

The term “critical quality attribute” or “CQA” as used herein means a physical, chemical, biological, or microbiological property or characteristic that should be within an appropriate limit, range, or distribution to ensure the desired product quality. CQAs are derived from a quality target profile and/or prior knowledge used to guide product and process development. Quality risk management can be used to prioritize the list of potential CQAs for subsequent evaluation. Relevant CQAs can be identified by an iterative process of quality risk management and experimentation that assesses the extent to which their variation can have an impact on the quality of the product.

The term “drug product” as used herein means a finished dosage form, for example, tablet, capsule, solution, etc., that contains an active drug ingredient generally, but not necessarily, in association with inactive ingredients. The term also includes a finished dosage form that does not contain an active ingredient but is intended to be used as a placebo. As used herein, the term “drug” and “pharmaceutical” include human drugs and veterinary drugs, and encompass both low molecular weight (i.e., small molecule) and biological drug products.

The term “excipient” (also “inactive ingredient”) as used herein means any component other than an active ingredient.

The term “finished product” as used herein means a product that comprises one or more ingredients. When the finished product is a pharmaceutical composition, for example, it may be in the form of a tablet, a capsule, a solution, etc. and will generally include an active ingredient in association with inactive ingredients.

The term “hybrid batch/continuous process” means a process composed of both batch and continuous unit operations. Examples include roller compaction and wet granulation.

The term “in process” material as used herein means any material fabricated, derived by chemical reaction, compounded or blended, that is produced for, and used in, the preparation of the product.

The term “lot” as used herein means a batch, or a specific identified portion of a batch, having an essentially uniform character and quality within specified limits; or in the case of a product produced by a continuous process, it is a specific identified amount produced in a unit of time or quantity in a manner that assures its having essentially uniform character and quality within specified limits. Definitions for both “batch” and “lot” are applicable to continuous processes.

The term “noncritical” process parameter” as used herein means a process parameter that does not have a direct impact on product quality.

The term “Probability of Acceptance value” or “Pa” as used herein means the likelihood that a process will produce product that meets an acceptance criteria for the required quality attribute.

The term “process parameters” or “operating parameters” as used herein mean the conditions under which a process is performed. Examples include, but are not limited to, temperature, pressure, flow rate, etc.

The term “process performance qualification” or “PPQ” as used herein means confirmation that the manufacturing process as designed is capable of reproducible commercial manufacturing.

The term “process validation” or “PV” as used herein means the collection and evaluation of data, from the process design stage through commercial production which establishes scientific evidence that a process is capable of consistently delivering quality product.

The term “quality” as used herein means the suitability of a substance or product for its intended use.

### Technical Risk Assessment Tool

The process validation life cycle approach involves technical risk assessment (TRA) throughout all stages of the product lifecycle. Referring to FIG. 2, technical risk assessment is typically used in step 203 of Process Design (Stage 1), step 206 of Process Qualification (Stage 2), and/or steps 209 and 211 of Process Monitoring (Stage 3) to avoid unexpected risks. Technical risk assessment is a significant part of change review. To be thorough, the technical risk assessment should encompass risk associated with formulation of the product, the process design, and scale-up of the associated commercial process.

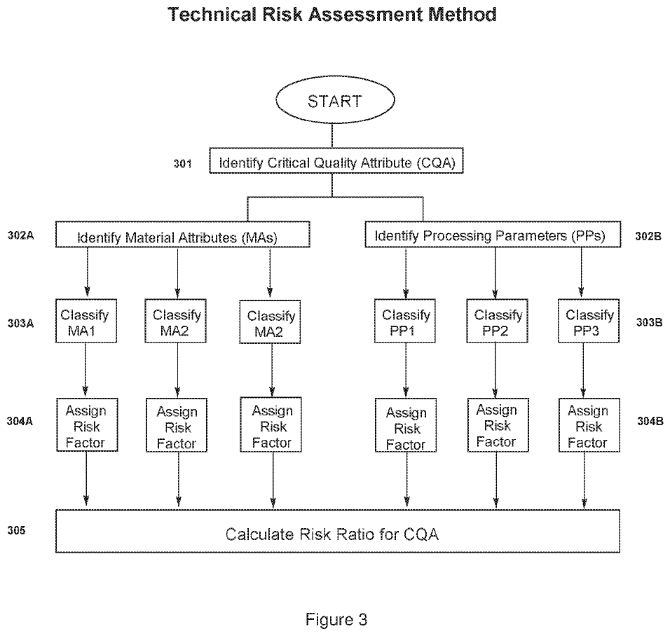

To facilitate the technical risk assessment, there is provided a method for analysis of a product manufacturing process. An example flowchart for the method is provided in FIG. 3, the method comprising: identifying a critical quality attribute (CQA) of a product manufactured by the process (301); identifying a plurality of material attributes (302A), a plurality of processing parameters (3026), or both, for the CQA, wherein each material attribute and/or processing parameter has a risk attribute for the critical quality attribute based on data; classifying each of the material attributes (303A) and/or processing parameters (303B), based on the associated risk attribute of the material attribute and/or processing parameter, for the CQA into a category of a plurality of categories, each category having an assigned risk; and determining, by a hardware computer processor, a risk ratio for the critical quality attribute (CQA) based on the classification of the material attributes and/or processing parameters into the categories (305).

In an embodiment, the critical quality attribute (CQA) is classified as a label claim CQA which has direct end user impact or a non-label claim CQA which has no direct end user impact. In an embodiment, the CQA is classified as a label claim CQA which has direct end user impact. In an embodiment, the label claim CQA comprises assay, concentration, content uniformity, dissolution, drug release, fill weight, or potency.

In an embodiment, the critical quality attribute (CQA) is classified as a non-label claim CQA which has no direct end user impact. In an embodiment, the non-label claim CQA comprises adhesion, assay per spray, blend uniformity, bulk density, cold flow, disintegration, droplet size, extractable volume, friability, hardness, integrity, osmolality, osmolarity, particle count, particle size distribution, particulate matter, peel, pH, shear, specific gravity, spray pattern, sterility assurance level, tack, thickness, torque, unit weight, viscosity, or weight variation.

In an embodiment, the risk ratio is based on a count of each of the product material attributes and/or processing parameters in each of the categories.

In an embodiment, the risk attributes comprise a risk attribute of whether the material attribute and/or processing parameter is critical or not critical to the product, a risk attribute of whether the material attribute and/or processing parameter has been evaluated or not evaluated, and a risk attribute of whether the material attribute and/or processing parameter has been mitigated or not mitigated.

In an embodiment, the categories include a first category for a material attribute and/or processing parameter with a critical risk attribute and a not evaluated risk attribute, a second category for a material attribute and/or processing parameter with a critical risk attribute and a not mitigated risk attribute, and a third category for a material attribute and/or processing parameter with a critical risk attribute and a mitigated risk attribute.

In an embodiment, the risk ratio is a sum of the count of material attributes and/or process parameters in the first and second categories divided by a sum of the count of material attributes and/or process parameters in the first, second and third categories.

In an embodiment, each of the categories is assigned a risk factor (304A and 304B in FIG. 3), at least two of the risk factors being different. In an embodiment, the risk factor is an integer ranging from 0 to 10.

In an embodiment, the categories are for a non-label claim CQA and two or more of the risk factors are different integers selected from the group consisting of 0, 1, 2, 3, 4, and 5. In an embodiment, the categories are for a label claim CQA and two or more of the risk factors are different integers selected from the group consisting of 0, 2, 4, 6, 8, and 10.

In an embodiment, a risk score is based on the count of each of the material attributes and/or processing parameters per category multiplied by the risk factor for that category.

In an embodiment, the method further comprises applying risk attributes to the material attributes and/or process parameters based on a decision tree. In an embodiment, the decision tree comprises applying to a material attribute and/or a processing parameter a risk attribute of whether the material attribute and/or processing parameter is critical or not critical to the CQA, applying to the material attribute and/or the processing parameter a risk attribute of whether the material attribute and/or processing parameter has been evaluated or not evaluated provided it has a critical or not critical risk attribute, and applying to the material attribute and/or the processing parameter a risk attribute of whether the material attribute and/or processing parameter has been mitigated or not mitigated provided it has an evaluated or not evaluated risk attribute.

In an embodiment, the method further comprises generating, by a hardware computer processor, a risk assessment chart for the critical quality attribute (CQA), the chart providing an indication of the category, for the CQA, for one or more of the material attributes and/or process parameters. In an embodiment, the risk assessment chart comprises an area chart, a bar chart, a bubble chart, a cone chart, a doughnut chart, a line chart, a Pareto chart, a pie chart, a radar chart, a scatter chart, a surface chart, a heat map, or a prioritization matrix.

In an embodiment, the method further comprises identifying at least one material attribute or process parameter from the plurality of material attributes and process parameters for optimization. In an embodiment, the method further comprises developing a control strategy for the product manufacturing process based on the risk ratio. In an embodiment, the method further comprises modifying the product manufacturing process based on the risk ratio.

In an embodiment, the critical quality attribute (CQA) is identified from a formulation process. In an embodiment, the material attributes and/or processing parameters are identified from a formulation process, a product development process, a product optimization process, or any combination thereof.

In an embodiment, the method comprises identifying the plurality of material attributes and wherein one of more of the product material attributes are of raw material.

In an embodiment, the method comprises identifying the plurality of process parameters and wherein one or more of the process parameters impact a critical quality attribute.

In an embodiment, the method comprises determining the risk ratio for a formulation, a product development process, a product optimization process, or any combination thereof. In an embodiment, the method comprises determining the risk ratio for a commercial process.

In an embodiment, the risk ratio for the critical quality attribute (CQA) is used to monitor the collection of raw data. In an embodiment, the risk ratio for the critical quality attribute (CQA) is used to determine the need for additional testing and/or need for designing an additional study.

In an embodiment, there is provided a system to analyze a product manufacturing process, the system comprising: a hardware computer processor; and computer readable program code, when processed by the processor, configured to: enable identification of a critical quality attribute (CQA) of a product manufactured by the process, enable identification of a plurality of material attributes, a plurality of processing parameters of the process, or both, for the CQA, wherein each material attribute and/or processing parameter has a risk attribute for the critical quality attribute based on data, classify each of the material attributes and/or processing parameters, based on the associated risk attribute of the material attribute and/or processing parameter, for the CQA into a category of a plurality of categories, each category having an assigned risk, and determine a risk ratio for the critical quality attribute (CQA) based on the classification of the material attributes and/or process parameters into the categories.

The following non-limiting embodiments and examples are provided to further illustrate the present description.

Application of the technical risk assessment can facilitate formulation and process development to ensure final product quality and reduce the risk of the Cost of Quality as part of Quality by Design (QbD) initiative according to International Conference on Harmonisation ICH Q8(R2) Pharmaceutical Development Guidance [ICH Harmonized Tripartite Guideline, Pharmaceutical Development Q8(R2), August 2009]. In an embodiment, the technical risk assessment is applied in Stage 1. But, it could be used in other Stages.

Formulation risk assesses the impact of material attributes in relation to the finished product CQAs. Prior knowledge, literature and experimental data are utilized to complete the formulation risk assessment. One example of formulation risk is the impact of disintegrant on dissolution.

Process risk assesses the impact of process parameters in relation to the finished product CQAs. Prior knowledge, literature, similar product/process data and experimental data are utilized to complete the process risk assessment. One example of process risk is the impact of compaction forces on dissolution.

Scale-up risk assesses the impact of scale-up factors and commercial process parameters on finished product CQAs. Prior knowledge, literature, similar product/process data and experimental data are utilized to complete the scale-up risk assessment. One example of scale-up risk is the impact of batch size on content uniformity.

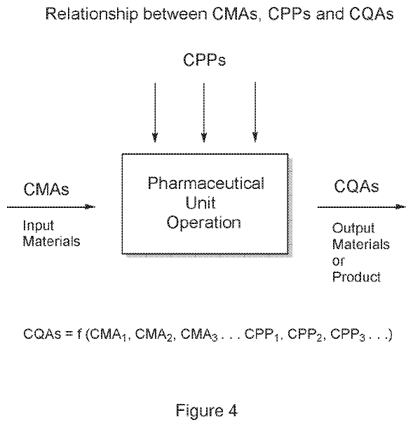

A first step in the process is to use a Product Development Report to identify critical material attributes (CMAs) and critical process parameters (CPPs) in order to establish the functional relationships that link critical material attributes (CMAs) and critical process parameters (CPPs) to critical quality attributes (CQAs). The relationship between CMAs, CPPs and CQAs is depicted in FIG. 4.

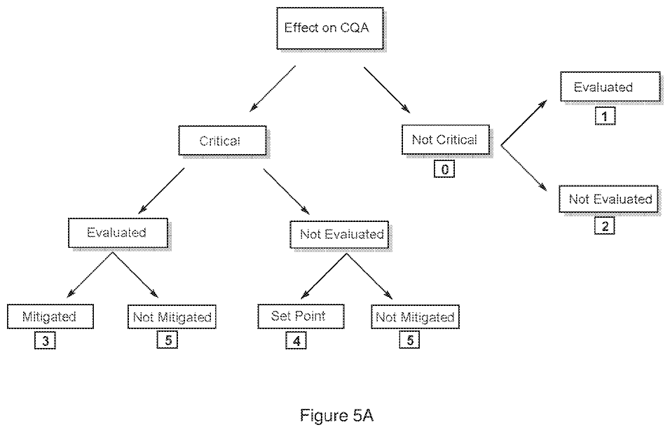

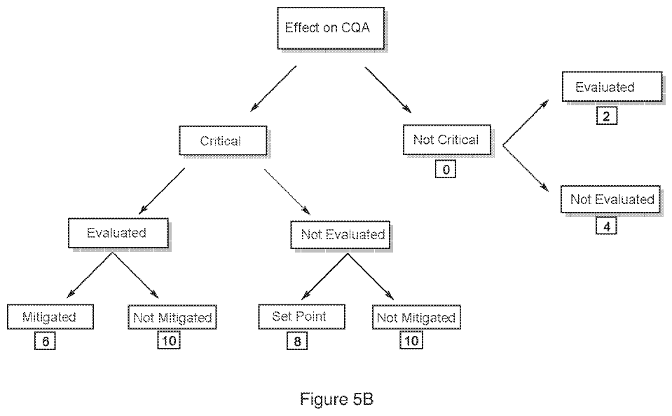

After the CMAs and CPPs have been identified, the impact of each CMA and CPP on the CQA is established. This is done by evaluating each variable using a decision tree, as shown in FIG. 5A and FIG. 5B.

In step 1 of the decision tree, the CMA or CPP is determined to be critical or not critical. The CMA or CPP is critical if one or more of the following conditions is met: (1) the CMA/CPP has a proven effect on the CQA; (2) the CMA/CPP is assumed to have an effect on the CQA; (3) the CMA/CPP was proven to be critical in design of experiment (DOE) or experimental trials; (4) the CMA/CPP was proven to be critical in a literature study; and/or (5) the CMA/CPP was found to have an impact on processability.

Step 2 of the decision tree relates to both critical and not Critical CMA/CPP. In step 2 of the decision tree, the CMA or CPP is categorized further as evaluated or not evaluated. That is, this categorizes whether the CMA or CPP has been considered in terms of, e.g., observation, measurement, etc.

Step 3 of the decision tree relates only to critical CMA/CPP. In step 3, critical CMA or CPP are categorized further still as mitigated, not mitigated, or controlled by a fixed set point.

In this way, each CMA and CPP linked to the CQA is evaluated using a decision tree to assign the CMA and CPP in a category.

Further, in an embodiment, a risk factor is assigned to the categories, wherein at least two of the categories have a different risk factor. In an embodiment, the risk factors are integers, wherein one category has a different risk factor integer value than another category. In an embodiment, the risk factor values depend on whether or not the CQA under review is a non-label claim CQA which has essentially no direct user impact (FIG. 5A) or a label claim CQA which has direct user impact (FIG. 5B). As described further below, the risk factors assigned for non-label claim CQA categories can be different than the risk factors assigned to label claim CQA categories.

For example, if the CQA under review is a non-label claim CQA which has essentially no direct end user impact, the risk factors can be assigned as shown in FIG. 5A. The risk factor assigned to each category is as follows: Not Critical (value 0), Not Critical/Evaluated (value 1), Not Critical/Not Evaluated (value 2), Critical/Mitigated (value 3), Critical/Controlled by Fixed Set Point (value 4), and Critical/Not Mitigated (5).

For example, if the CQA under review is a label claim CQA which has direct end user impact, the risk factors can be assigned as shown in FIG. 5B. The risk factor assigned to each category is as follows: Not Critical (value 0), Not Critical/Evaluated (value 2), Not Critical/Not Evaluated (value 4), Critical/Mitigated (value 6), Critical/Controlled by Fixed Set Point (value 8), and Critical/Not Mitigated (value 10).

The criticality of each variable can be shown visually in a heat map. The heat map is a colored representation of data wherein categories having differing risk factors are represented as different colors. For example, the colors for the categories in the heat map may be as follows: Critical/Not Mitigated (red), Critical/Not Evaluated (dark orange), Critical/Mitigated (orange), Not Critical/Not Evaluated (yellow), Not Critical/Evaluated (light green), and Not Critical (dark green). Optionally, the heat map does not display the Not Critical category having a risk factor of zero.

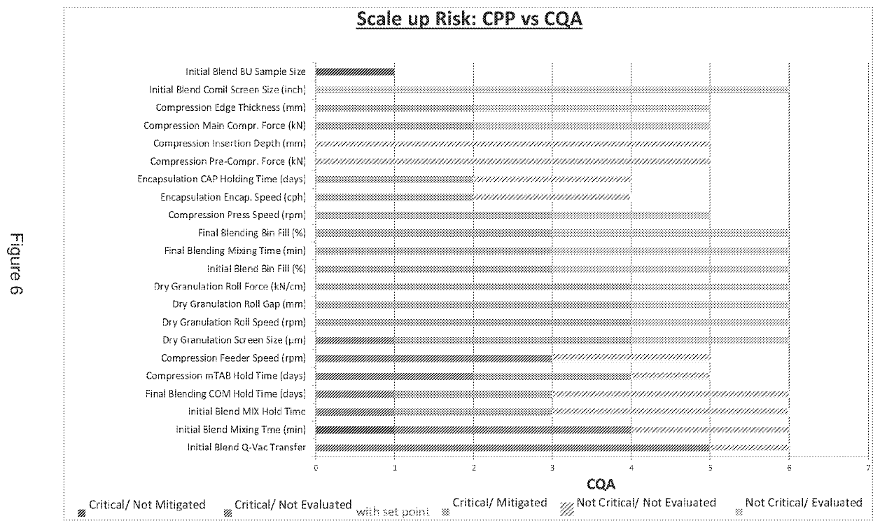

FIG. 6 shows an example heat map for scale-up risk. In this particular example, the category Not Critical has a risk factor of zero and is not shown on the heat map.

The data from the decision tree analysis can be tabulated and used to calculate a risk ratio and risk score. For example, the data from the scale-up risk analysis shown visually in the heat map in FIG. 6 is presented in Table 1 below.

TABLE 1
Tabulated Data for Scale-up Risk

| | Count of | | |
|---|---|---|---|
| | CMA/CPP | | Risk Score |
| | Factors in Each | Risk | for Each |
| Category | Category | Factor | Category |
| Not Critical | 0 | 0 | 0 × 0 = 0 |
| Not Critical/Evaluated | 31 | 1 | 31 × 1 = 31 |
| Not Critical/Not | 26 | 2 | 26 × 2 = 52 |
| Evalvated | | | |
| Critical/Mitigated | 41 | 3 | 41 × 3 = 123 |
| Critical/Not Evaluated | 16 | 4 | 16 × 4 = 54 |
| with Set Point | | | |
| Critical/Not Mitigated | 2 | 5 | 2 × 5 = 10 |

| | |
|---|---|
| Total Risk Score | 31 + 52 + 126 + 64 + 10 = 280 |
| Risk Ratio | (16 + 2)/(41 + 16 + 2) = 0.31 |

In an embodiment, the risk ratio is a sum of the count of material attributes and/or process parameters under the categories Critical/Not Evaluated (with Set Point) and Critical/Not Mitigated divided by the sum of the count of material attributes and/or process parameters under the categories Critical/Mitigated, Critical/Not Evaluated (with Set Point), and Critical/Not Mitigated.

Thus, a risk ratio for the process can be calculated per the equation shown below:

$$Risk Ratio = \frac{\begin{matrix}Sum of Count of CMA \text{/} CPP Factors \\ ( Critical \text{/} Not Evaluated with Set Point + \\ Critical \text{/} Not Mitigated )\end{matrix}}{\begin{matrix}\begin{matrix}Sum of Count of CMA \text{/} CPP Factors \\ ( Critical \text{/} Mitigated +\end{matrix} \\ Critical \text{/} Not Evaluated with Set Point + \\ Critical \text{/} Not Mitigated )\end{matrix}}$$

The calculated risk ratio for the process can then be assigned a risk level according to Table 2 below.

TABLE 2
Risk Level

| Risk Ratio | Risk |
|---|---|
| Less than 0.25 | Low Risk |
| 0.25-0.75 | Medium Risk |
| Higher than 0.75 | High Risk |

Further, a risk score for the process can be determined as the sum of the risk scores for the categories, wherein the risk score for each category is the count of material attributes and/or process parameters in the category multiplied by the associated risk factor. Table 1 above shows an example calculation of the risk score for the process. Like the risk ratio, the heat score can be evaluated against one or more thresholds.

Based on the heat map, risk ratio, and/or heat score for each stage, top CMAs and CPPs can be selected for optimization of the process to minimize/eliminate the risk identified. Following optimization, the technical risk assessment is repeated until, for example, the risk ratio is reduced to medium risk or low risk. The above method is applicable for determining formulation risk, functionality design risk, process risk, scale-up risk, and/or risk associated with product lifecycle changes.

Thus, this technique and tool can provide one or more of the following example advantages. This technique and tool can be used to develop the process flow, and simulate impacts to the process. This technique and tool can quantify risk for the purpose of scale-up, process validation, and/or change evaluation, and make decision making faster. This technique and tool can provide an objective measure of risk and can provides scientific justification for matrixing, bracketing of strengths, and evaluation of similar products/processes. Thus, this technique and tool can reduce or eliminate the need for additional batches for product development and/or validation. This technique and tool can capture current risk and in a way that the assessment can be updated continuously. Thus, additional time for rework/repeat risk assessment can be reduced or eliminated. This technique and tool can, based on FDA quality matrix draft guidance, extend FDA site audit cycle (e.g., from every 3 years to 5 years). This technique and tool can, in line with the 2011 FDA Guidance, systematically evaluate data and scientifically establish evidence that a process is capable of consistently delivering quality product. This technique and tool can make the process “right the first time” so as to avoid unexpected risks/issues.

### Method to Determine Minimum Number of Performance Qualification Lots

The process validation life cycle approach typically involves a determination of a number of performance qualification lots to assess whether the product manufacturing process will work as expected, Referring to FIG. 2, the method to determine the minimum number of performance qualification lots can be used in Step 204 of Process Design (Stage 1), Step 207 of Process Qualification (Stage 2) or Step 209 of Process Monitoring (Stage 3).

However, current methods to determine the number of performance qualification lots can be inadequate. The number of lots for performance qualification is usually determined by arbitrary and subjective assessment (for example, a number of lots is chosen that is expected to be sufficient based on experience) without distinguishing variability within lots and between lots. An improved method would use previously collected product information (e.g., data collected from Stage 1 lots for Stage 2 PPQ and/or historical lot-to-lot process information) to provide a scientific- and risk-based projection of the number of lots for performance qualification (e.g., Stage 2 PPQ).

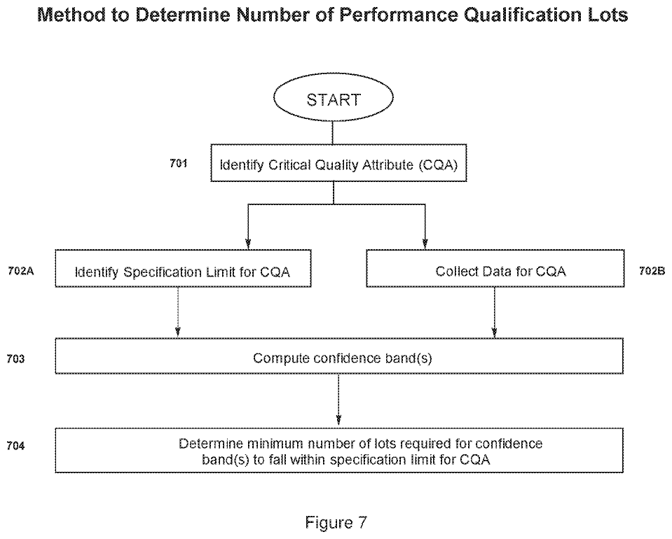

One aspect is a method to determine a minimum number of lots needed for performance qualification and/or verification of a product manufacturing process. A flowchart for the method is provided in FIG. 7, the method comprising: identifying a critical quality attribute (CQA) of a product manufactured by the process (701); identifying a specification limit for the CQA (702A); based on data collected for the CQA of a plurality of units of the product manufactured by the process (702B), computing, by a hardware computer processor, one or more confidence bands for a plurality of statistical measures of the data collected (703); and determining a minimum number of lots required for the one or more confidence bands to fall within the specification limit for the CQA (704).

In an embodiment, the minimum number of lots is for qualification and the qualification is process performance qualification, product performance qualification, method performance qualification, supplied material qualification, equipment qualification, facility qualification, or utility qualification. In an embodiment, the minimum number of lots is for verification and the verification is design verification or process verification.

In an embodiment, the data collected for the CQA is from development lots, lots of a similar process, lots of a similar product, or any combination thereof. In an embodiment, the data collected for the CQA is from lots of a similar process and the process is similar based on product strength, equipment type, a testing property, or any combination thereof and/or the data collected for the CQA is from lots of a similar product and the product is similar based on product strength, equipment type, a testing property, or any combination thereof.

In an embodiment, the method further comprises modifying the number of lots needed to qualify and/or verify a process based on data collected from the product manufacturing process. In an embodiment, the method further comprises manufacturing additional units of the product provided data collected from the product manufacturing process meets an acceptance criterion. In an embodiment, the method further comprises designating units of the product for sale or consumption provided data collected from the product manufacturing process meets an acceptance criterion.

In an embodiment, determining the minimum number of lots comprises determining the minimum number of lots required for the confidence band to fall within the specification limit for the CQA.

In an embodiment, the CQA is classified as a label claim CQA which has direct end user impact or a non-label claim CQA which has essentially no direct end user impact

In an embodiment, the data collected comprises intra-lot variability data for the CQAs. In an embodiment, intra-lot data is collected from lots produced for the purposes of a clinical trial, a governmental agency submission/registration study, a stability study, a process scale-up study, a method validation study, or an equipment capability study.

In an embodiment, data collected comprises lot-to lot performance data for the CQA. In an embodiment, the lot-to-lot performance data is estimated using lot to lot data collected from a similar product category where similar is defined as a similar label claim or a similar manufacturing process depending on process knowledge and data analysis.

In an embodiment, the plurality of statistical measures comprises mean and standard deviation.

In an embodiment, the confidence band is derived from:

$$\begin{matrix}μ = \overline{x} \pm {t}_{( \frac{a}{2} n - 1 )} \frac{s}{\sqrt{n}} & \\ and \text{/} or & \\ s \sqrt{\frac{n - 1}{{𝒳}_{( n - 1 , 1 - \frac{a}{2} )}^{2}}} \leq σ \leq s \sqrt{\frac{n - 1}{{𝒳}_{( n - 1 , \frac{a}{2} )}^{2}}} & \end{matrix}$$

wherein
μ=underlying population mean
σ=underlying standard deviation
n=the number of lots
x̅=measured mean
s=measured standard deviation
α=probability
t=t-distribution
χ²=chi-squared distribution.

In an embodiment, the method further comprises repeating the computing and determining for each of a plurality of critical quality attributes (CQA) of the product, a minimum number of lots required for a respective confidence band to fall within the specification limit for each CQA. In an embodiment, the method further comprises selecting from a minimum number of lots for each CQA, the highest minimum number as the required number of lots needed for qualification and/or verification of the product manufacturing process.

In an embodiment, computing the one or more confidence bands uses a formula derived from single-stage acceptance criteria for the CQA. In an embodiment, computing the one or more confidence bands uses a formula derived from multi-stage acceptance criteria for the CQA. In an embodiment, computing the one or more confidence bands comprises running a statistical simulation of the product manufacturing process. In an embodiment, the statistical simulation comprises a Monte Carlo simulation.

In an embodiment, the method further comprises determining whether the data collected has a normal or non-normal distribution and determining the one or more confidence bands based on data that has a normal distribution. In an embodiment, determining whether the data collected has a normal or non-normal distribution comprises performing statistical normality tests such as the Anderson-Darling normality test.

In an embodiment, the method is used to monitor the collection of raw data. In an embodiment, the method is used to determine the need for additional testing.

In an embodiment, the method further comprises comprising modifying the product manufacturing process based on data collected from a minimum number of lots of the product manufacturing process. In an embodiment, the method further comprises manufacturing additional units of the product provided data collected from a minimum number of lots of the product manufacturing process meets an acceptance criterion.

In an embodiment, there is provided a method of processing data collected from a product manufacturing process, the data relating to a critical quality attribute (CQA) of a product manufactured by the process, the method comprising: computing, based on data collected, a series of confidence bands, each confidence band being for a different number of lots of the product manufactured by the process; determining whether each confidence band falls within a specification limit for the CQA; selecting a minimum number of lots required for the confidence band to fall within the specification limit for the CQA; and selecting from a minimum number of lots for each CQA the highest minimum number as the required number of lots needed for qualification and/or verification of the product manufacturing process.

In an embodiment, there is provided a system to determine the number of lots needed for performance qualification and/or verification of a product manufacturing process, the system comprising: a hardware computer processor; and computer readable program code, when processed by the processor, configured to: enable identification of a critical quality attribute (CQA) of a product manufactured by the process; enable identification of a specification limit for the CQA; based on data collected for the CQA of a plurality of units of the product manufactured by the process, compute one or more confidence bands for a plurality of statistical measures of the data collected, and determine a minimum number of lots required for the confidence band to fall within the specification limit for the CQA.

In an embodiment, there is provided a system to process data collected, from a product manufacturing process, relating to a critical quality attribute (CQA) of a product, the system comprising: a hardware computer processor; and computer readable program code, when processed by the processor, configured to: compute, based on data collected, a series of confidence bands, each confidence band being for a different number of lots of the product manufactured by the process; determine whether each confidence band falls within a specification limit for the CQA; select a minimum number of lots required for the confidence band to fall within the specification limit for the CQA; and select from a minimum number of lots the highest minimum number as the required number of lots needed for qualification and/or verification of the product manufacturing process.

Further details can be found in the paper by one or more of the inventors titled A. Pazhayattil et al., “Stage 2 Process Performance Qualification (PPQ): a Scientific Approach to Determine the Number of PPQ Batches”, AAPS PharmSciTech (Sep. 8, 2015), which is incorporated herein in its entirety by reference.

The following non-limiting embodiments and examples are provided to further illustrate the present description,

### Determination of the Number of Batches

In a batch manufacturing process for pharmaceutical products, process performance lots are sometimes referred to as process performance qualification (PPQ) batches. They are batches used to qualify and/or verify the process. Use of the phrase “batch” or “PPQ batches” in the following embodiments and examples is illustrative; it does not limit the scope of the disclosure herein to, for example, a batch manufacturing process or to process performance qualification.

So, an approach and tool to determine and justify a minimum number of batches that should be evaluated (e.g., for Stage 2 PPQ) is described. Desirably, the approach is in compliance with the 2011 FDA Guidance. The described statistical tool projects the number of batches that should establish sufficient scientific evidence that the process is robust and will consistently deliver quality products. In an embodiment, the approach uses previously collected product specific information (e.g., data generated from Stage 1 batches produced for the purpose of a clinical trial, a governmental submission/registration, a stability study, and/or a process scale-up/demonstration effort), and historical batch-to batch process information (e.g., typical variability observed for this product/process type, e.g., based on active content) across one or more critical quality attributes (e.g., content uniformity, assay, and/or dissolution) to provide a science- and risk-based projection of the number of batches required for qualification and/or verification (e.g., Stage 2 PPQ).

In an embodiment, the approach and tool determines a minimum number of batches for which a projected confidence interval of the product's one or more (desirably plurality of) critical quality attributes resides completely and readily within a desired specification. That is, based on “current” information, a number of batches that upon evaluation should provide sufficient data so that a statistically confident conclusion of the product's one or more critical quality attributes can be achieved. In an embodiment, this involves estimating a confidence interval for each number of potential PPQ batches based on previously collected product specific data (i.e., the magnitude of the “within” or intra-batch statistics) and historical evidence of batch-to-batch variability for comparable products (i.e., “between” or inter-batch). Per this approach, the projected number of PPQ batches is determined where the entire confidence interval resides within the specification limits.

In an embodiment, for a product quality attribute to be tested to comply with a specification, its tested mean should be as close as possible to the center of the specification and its standard deviation should be as minimal as possible under the assumption of normal distribution. So, based on this assumption, the approach and tool, in an embodiment, uses a confidence interval of the product quality attribute measurements that is a combination of the confidence interval of the process mean and the confidence interval of the process standard deviation.

Because each specific quality attribute is framed differently, often with distinct requirements, the form of the equations used to determine confidence intervals is tailored per quality attribute and/or number of acceptance stage criteria as discussed further below.

So, in an embodiment, a generalized approach of determining the number of batches is to obtain, for a critical quality attribute, product specific process mean information from, e.g., Stage 1 data. Then, between-batch and within-batch variation information is determined or estimated. Then, one or more overall confidence bands can be computed for the product based on the number of batches (e.g., n=1, 2, 3, 4, etc.) and based on, for example, the confidence of mean and confidence of variation. The one or more confidence bands are compared with a specification and the minimum number of batches for performance qualification and/or verification can be selected as the lowest number of batches for which its confidence band falls within the specification. This can be repeated for one or more additional critical quality attributes. Thus, a minimum number of batches can determined for each of the critical quality attributes. In that case, a minimum number of batches for performance qualification and/or verification could the maximum number of batches selected from minimum numbers of batches for the plurality of critical quality attributes.

The total or overall variability of a process can be represented as a summation of individual component variation. This may be mathematically denoted as:

sₜₒₜₐₗ²=s_{batch-batch}²+s_{intrabatch}²+s_{sampling}²+s_{analytical}²+ . . . (1)

Total variation is comprised of variation derived from batch-to-batch, intra-batch product, sampling, and analytical variability sources. Such sources of variation are typical for a process. In general, Stage 1—Process Design provides an assessment of most variation sources with a notable exception of batch-to-batch (between or inter-batch) variability. Thus, data from Stage 1 provides a reasonable measure of product intra-batch performance. However, the batch-to-batch variability can't be properly assessed until several batches of product are produced and analyzed. To approximate this component, it is reasonable to assert that a similar process/product will exhibit similar batch-to-batch characteristics and tabulated evidence from historical records can provide a good estimate (See Example: Determination of Batch-to-Batch Variability).

The deduction of the true underlying population parameters from a limited amount of sampling can be statistically determined through the use of confidence bands. In an embodiment, the confidence band for mean is defined by the following equation:

$$μ = \overline{x} \pm {t}_{( \frac{α}{2} , n - 1 )} \frac{s}{\sqrt{n}} \qquad ( 2 )$$

and for standard deviation is defined by the following equation:

$$s \sqrt{\frac{n - 1}{{χ}_{〈 n - 1 , 1 - \frac{α}{2} 〉}^{2}}} \leq σ \leq s \sqrt{\frac{n - 1}{{χ}_{( n - 1 \frac{α}{2} )}^{2}}} \qquad ( 3 )$$

wherein,

μ=underlying population mean

σ=underlying standard deviation

n=the number of sample groups

x̅=measured mean

s=measured standard deviation

α=probability

t=t-distribution

χ²=chi-squared distribution.

Note that chi-square and t-distribution are a function of the number of sample groups (n).

Equation 3 describes how the confidence band of the variance (in that case, of the mean and standard deviation) improves with increasing number of acquired batches. Thus, in an embodiment, the number of batches (e.g., minimum number of batches) needed for performance qualification and/or verification is the number of batches when the confidence interval of a plurality of product quality attribute measurements, which is, e.g., a combination of the confidence interval of the process mean and the confidence interval of the process standard deviation, resides completely in a specification range.

Note that an overall process standard deviation is composed of within-batch and between-batch variations. Within-batch variation is product specific and can be obtained from, e.g., Stage 1 batches produced for the purpose of a clinical trial, a governmental agency submission/registration, a stability study, and/or a process scale-up/demonstration study. Between-batch variability for a specific product is usually not available before the execution of Stage 2 manufacturing. Thus, between-batch variability can be estimated using historical batch-to batch process information (e.g. typical variability observed for this product/process type based on active content).

Thus, this technique and tool can provide one or more of the following example advantages. It can provide a justification to, e.g., a governmental agency for the number of batches selected. The technique and tool could prevent excessive/unnecessary or insufficient data collection during Stage 2. The technique and tool can be used for process, analytical, equipment, systems, and/or utility-performance qualifications. The technique and tool can be applied to various different kinds of products and at different stages (e.g., determine Stage 3A Continued Process Verification batches). The technique and tool can be used predictively to further gain confidence in process verification. The technique and tool can be used to develop and/or justify sampling plans for product development, scale-up and/or investigations. The technique and tool can incorporate Stage 1 performance and historical process understanding into an overall risk assessment. As described below, the technique and tool can be flexible, e.g., can be customized for various types of critical quality attributes. The technique and tool can distinguish and characterize within batch and between batch variability. The technique and tool can analyze various critical quality attributes (e.g., assay, content uniformity, dissolution and/or strengths, which are often components of the label claim).

Because each specific quality attribute is framed differently, often with distinct requirements, the form of the equations used to determine confidence intervals is tailored per quality attribute and/or number of stages of acceptance criteria.

### Example: CQA Having One-Stage Acceptance Criteria—Assay

There is typically only one assay sample (i.e. a composite of at least ten dosage units) analyzed per batch. Thus the impact of intra-batch variation on assay is considered less significant in assessing the overall variation. Referring to equations (2) and (3), the form of the confidence limits for a progressive number of batches for assay can become:

$${\overline{x}}_{{N}_{B}}^{hi / lo} = {\overline{x}}_{0} \pm {t}_{( 1 - \frac{α}{2} , {N}_{B} - 1 )} * \frac{{s}_{B - B} * \sqrt{\frac{( {N}_{B} - 1 )}{{χ}_{( \frac{a}{2} , {N}_{B} - 1 )}^{2}}}}{\sqrt{{N}_{B}}} \qquad ( 4 )$$

wherein x̅₀ represents the average of assay values determined during Stage 1 efforts, s_{B-B} represents batch-to-batch standard deviation of comparable processes determined from historical information (as with content uniformity data (described below), pre-existing batch data can be used to determine the inter-batch variability), and N_{B} represents the number of batches (e.g., which is progressively increased to find a minimum number of batches).

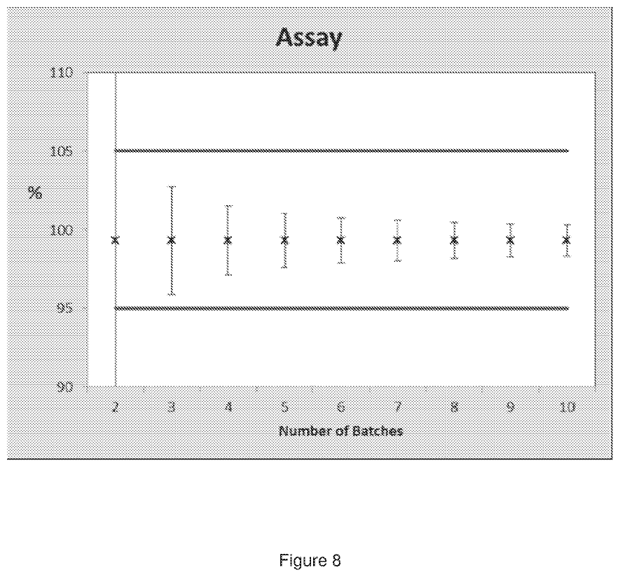

Because assay has a two sided specification (typically 95 to 105%), both the upper and lower confidence interval limits must reside within the specification requirement to project the number of batches that will be sufficient for evaluation. As illustrated in an example as presented in FIG. 8, three Stage 2 PPQ batches should be sufficient to assure a robust process for the data underlying that chart.

Similar techniques could be applied to other attributes with a one-stage acceptance criteria.

### Example: CQA Having Two-Stage Acceptance Criteria—Content Uniformity

Section <905>“Uniformity of Dosage Units” in the United States Pharmacopeia provides guidance on assessing the consistency of dosage units [The United States Pharmacopeia Convention, General Chapter <905> Uniformity of Dosage Units. The United States Pharmacopeia and The National Formulary, Dec. 1, 2011 (“USP <905>”), incorporated herein by reference in its entirety]. The uniformity of dosage units can be demonstrated by either of two methods, content uniformity or weight variation. The test for content uniformity of preparations presented in dosage units is based on the assay of the individual content of drug substance(s) in a number of dosage units to determine whether the individual content is within the limits set.

For the content uniformity test, the guidance provides a two stage acceptance criteria through computation of an acceptance value (AV). USP <905> describes multiple cases depending on the measured average of the sampled units. The general form of the AV equation is:

$$\text{?} \text{?} \text{indicates text missing or illegible when filed} \qquad ( 5 )$$

[symbol]

wherein M is a reference value, x̅ is the mean of individual contents expressed as a percentage of the label claim, k is the acceptability constant (e.g., if sample size n=10, then k=2.4 and if sample size n=30, then k=2.0), and s is the sample standard deviation.

Note that while USP <905> provides more details, this example only describes a simplified case where the target dosage value is ≤101.5% and the measured mean is between 98.5% and 101.5%. The simplified example is presented here for illustration and clarity only. For acceptance stage L1, the simplified equation becomes, AV=2.4 s. Using equations (1) and (3), the following equation can be derived to determine the AV upper confidence limit for a progressive number of batches (N_{B}).

$${s}_{( {N}_{B} )}^{h i} = \sqrt{{\{ {s}_{B - B} * \sqrt{\frac{( {N}_{B} - 1 )}{{χ}_{( \frac{α}{2} , {N}_{B} - 1 )}^{2}}} \}}^{2} {\{ {s}_{0} * \sqrt{\frac{( {N}_{0} - 1 )}{{χ}_{( \frac{α}{2} , {N}_{0} - 1 )}^{2}}} \}}^{2}} \qquad ( 6 )$$

wherein s_{B-B} represents the batch-to-batch standard deviation of comparable processes determined from historical information; s₀ represents the standard deviation from previously data collected on the specific process (e.g. Stage 1 data); N₁ represents the number of data points used to determine s₀; and α represents the desired confidence for the determination (typically 0.05).

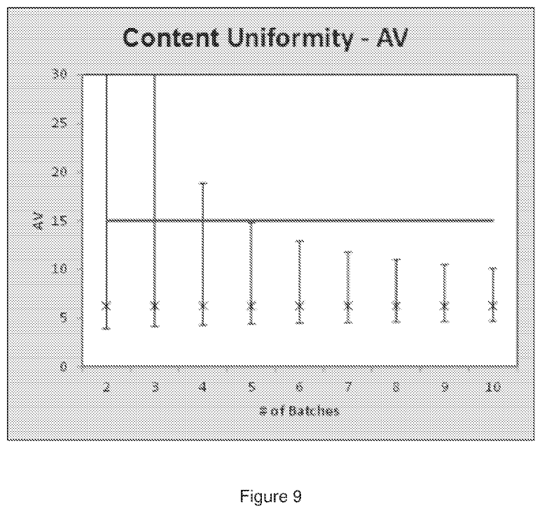

A confidence band is then estimated for each number of potential PPQ batches based on previously collected product specific data (e.g., the magnitude of the “within” or intra-batch statistics) and historical evidence of batch-to-batch variability for comparable products (i.e. “between” or inter-batch). Per this approach, the projected number of PPQ batches is determined where the entire confidence interval resides within the specification limits. As illustrated in an example as presented in FIG. 9, the suggested number of Stage 2 PPQ batches is no less than five to ensure a robust process for the data underlying that chart.

Similar techniques could be applied to other attributes with a two-stage acceptance criteria.

### Example: CQA Having Three or More-Stage Acceptance Criteria—Dissolution

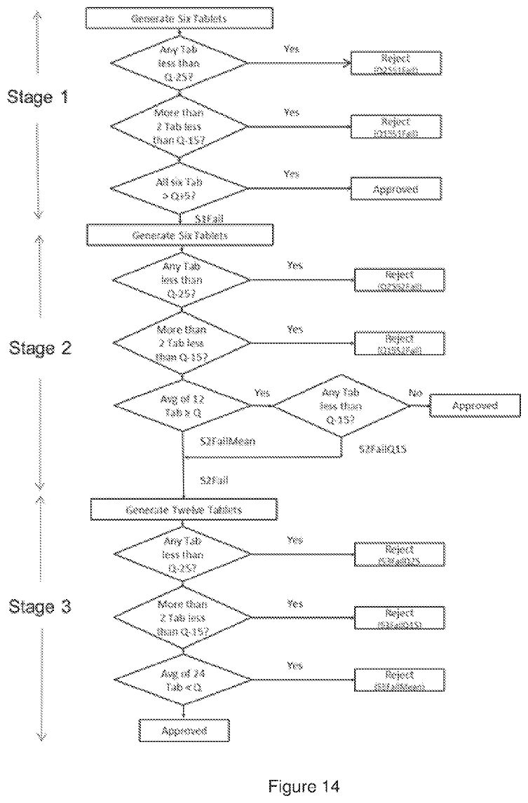

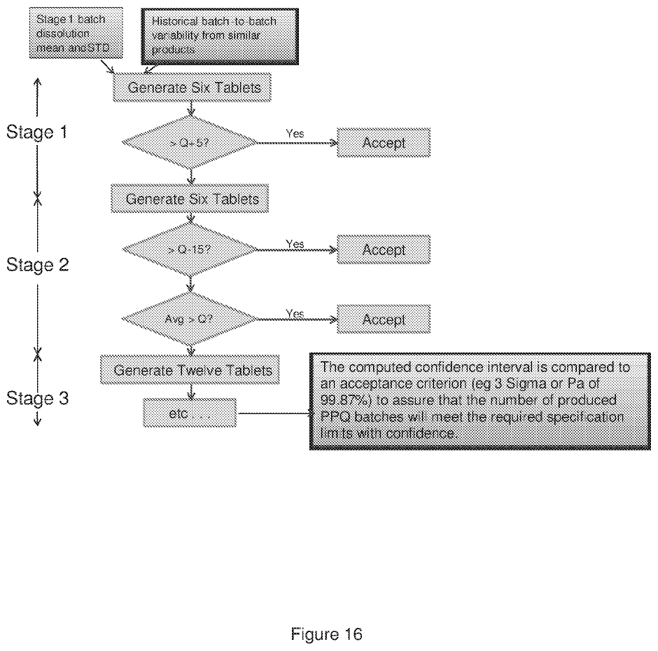

Dissolution is a considerably more complicated analysis than assay or content uniformity. For example, immediate release product dissolution follows a complicated three stage acceptance criteria. The chance that a particular product will meet the overall stage-wise criteria can be defined by the Probability of Acceptance (Pa). The stage-wise rules essentially create a complicated equation that transforms the mean and standard deviation of the acquired dissolution data into an acceptance probability. Among other approaches, this equation can be solved using a Monte Carlo computation wherein the results are stored into a series of lookup tables (Dissolution Monte Carlo Transformation). An example flow chart describing a Monte Carlo simulation process for dissolution is presented in FIG. 16. The process is run through different combinations of parameters—Q, mean, standard deviation (STD), and so forth—selected in a Monte Carlo fashion. An example flow chart describing a Monte Carlo simulation process for dissolution is presented in FIG. 14.

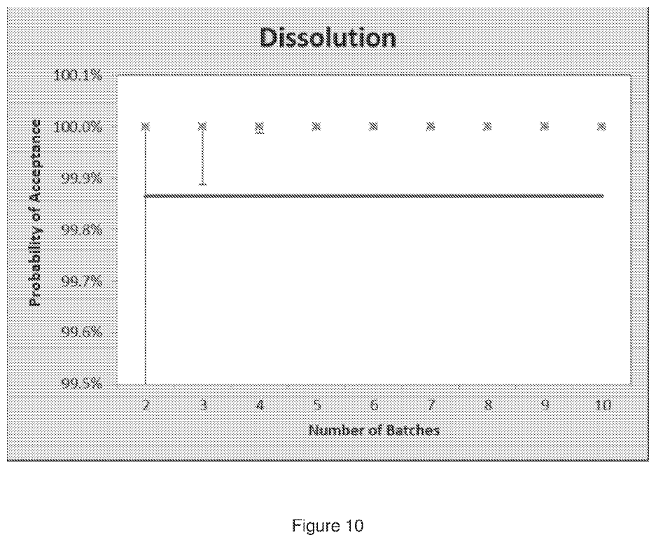

Despite the complexity in the dissolution acceptance probability equation, the defined approach to determining the suggested number of batches based on dissolution data is similar to that described for content uniformity. The specific product dissolution statistics are combined with historical batch-to-batch variability from similar products to determine confidence limits for both the mean and standard deviation statistics using equations (1)-(3). The upper and lower limits are used appropriately along with the Dissolution Monte Carlo Transformation to determine a probability of acceptance. The computed confidence interval is compared to an acceptance criterion to assure that the number of produced PPQ batches will meet the required specification limits with confidence. An example plot wherein the dissolution criterion is set at a 3 sigma level (acceptance probability greater than 99.87%) is shown in FIG. 10. This example suggests a minimum of three batches for Stage 2 PPQ for dissolution for the data underlying that chart.

Similar techniques could be applied to other attributes with a three or more-stage acceptance criteria.

### Example: Determination of Batch to Batch Variability

A factor in the overall determination of suggested number of PPQ batches is the batch-to-batch (or between batch) variability. Prior to the PPQ evaluation, this factor for the particular product has yet to be determined. However, data of comparable products and/or processes provide a reasonable indication of the anticipated batch-to-batch variability. The magnitude of the batch-to-batch variability is potentially dependent on several different factors; one factor in particular is the API content or product label claim.

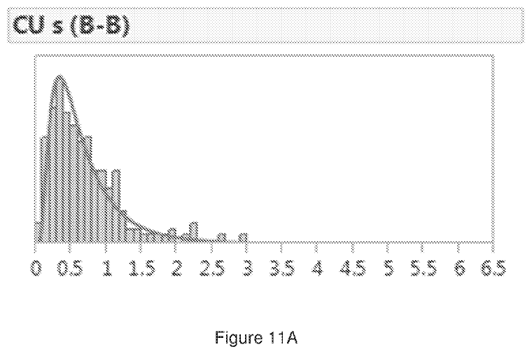

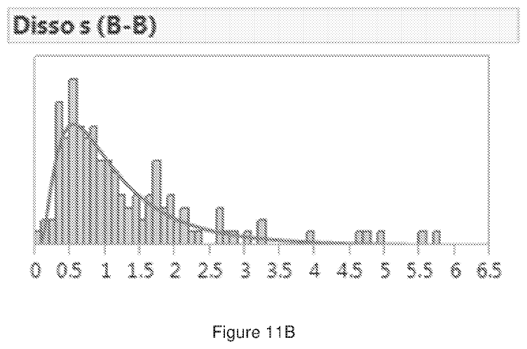

To gain an understanding of batch-to-batch variability, historical content uniformity and dissolution data from over 700 individual batches and approximately 100 distinct molecules was compiled. The batch-to-batch variability was extracted from each campaign by separating the intra-batch variability from the overall total campaign variability (equation (1)). The distribution of each campaign batch-to-batch standard deviation is shown in FIG. 11A for content uniformity (CU) and in FIG. 11B for dissolution (Disso). This data exhibits a profile comparable to a chi square distribution (i.e. non-normal and skewed), that is a typical distribution profile expected for a collection of standard deviation data.

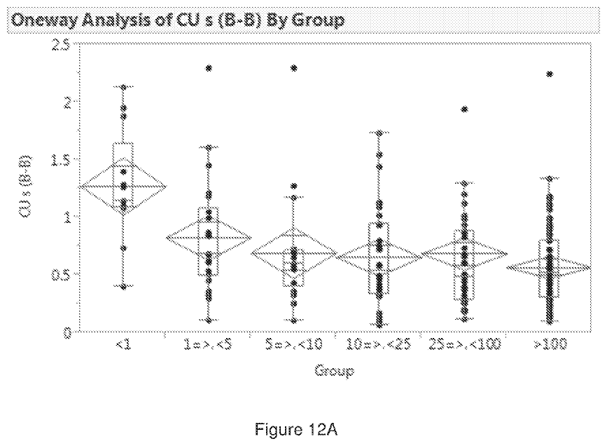

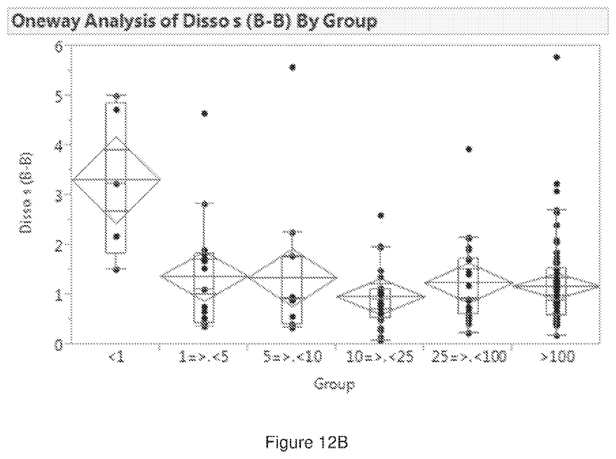

The data was segregated and analyzed to assess the relative influence of several factors (e.g. manufacturing process, strength, batch size, etc.). A particular factor that appeared correlated to batch-to-batch variability was the product active content or strength. FIGS. 12A and 12B show that the batch-to-batch variability for both content uniformity (CU) and dissolution, as a function of product strength significantly increases for low strength products (<1 mg). Thus, one relevant model input factor for batch-to-batch variability is active content or strength.

Summary data from the historical evidence, such as that above, can be stored into reference tables and used as a reasonable approximation of the batch-to-batch component of variation used in justifying the number of batches that should be evaluated during PPQ to provide reasonable confidence that the evaluated process is robust. Once these PPQ batches are manufactured and tested, it is prudent to compare the approximation of the batch-to-batch variation with the truly observed PPQ batch-to-batch variation.

### Probability of Acceptance (Pa) Analysis

The acceptance criteria for pharmaceutical products are often multi-level and more intricate than other industries. Traditional statistical process control strategies (e.g., Cₚₖ, Pₚₖ, control charts) may give erroneous indications or fall short in providing a suitable assessment of product risk. A more applicable analysis method is desired to provide a reliable understanding of the ability to fulfill the requirements for the quality attributes.

Thus, there is provided a method and tool that provides a scientifically unbiased approach towards understanding how variation impacts the likelihood that a manufacturing process will produce product that meets the required quality attribute acceptance criteria. This method and tool, referred to herein as Probability of Acceptance (Pa) analysis, is designed to provide a probability that a future produced batch will meet a specification acceptance criteria. As will be appreciated, the probability can be specified in any number of ways, such as a percentage, a decimal or fraction, etc.

The Probability of Acceptance (Pa) can provide a direct and readily understandable indication of product risk. Pa is designed to directly relate to the assurance that a future batch will meet the required specification. Referring to FIG. 2, the Probability of Acceptance analysis can be used, for example, in Step 205 of Process Design (Stage 1), Step 208 of Process Qualification (Stage 2), and/or Steps 210 and 211 of Process Monitoring (Stage 3).

In contrast to, e.g., Cpk, which typically provides information about a future single unit, Probability of Acceptance (Pa) is designed to, e.g., provide a probability that a future produced batch will meet the specification acceptance criteria. The resultant Pa outcome is considerably more distinctive than a capability index. It is challenging to understand the implications of a process with a Cpk=1.28. However, the meaning of a 99.93% probability that a future batch will meet the requirements is easily understood. In an embodiment, Pa is designed to directly relate to the assurance that a future batch will meet the required specification.

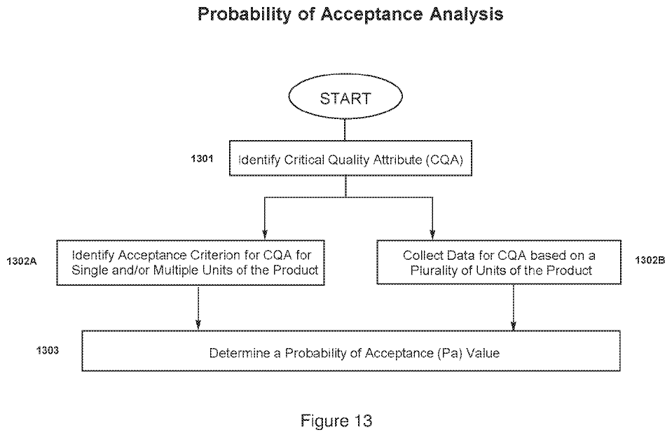

An example flowchart for the Probability of Acceptance analysis is provided in FIG. 13. This analysis provides a method for risk assessment in a product manufacturing process, the method comprising: identifying a critical quality attribute (CQA) of a product manufactured by the process (1301); identifying an acceptance criterion for the CQA for single and/or multiple units of the product manufactured by the process (1302A); based on data collected for the CQA of a plurality of units of the product manufactured by the process (1302B), determining, by a hardware computer processor, a Probability of Acceptance (Pa) value that specifies the probability that the acceptance criterion for a future unit of the product will meet or cross, or will not meet or not cross, a threshold defined for the acceptance criterion (1303).

In an embodiment, the method further comprises modifying the product manufacturing process based on the Probability of Acceptance (Pa) value. In an embodiment, the method further comprises modifying equipment or a test method. In an embodiment, the method further comprises modification of the product manufacturing process at Process Validation Stage 1, Stage 2, Stage 3, or any combination thereof.

In an embodiment, the method further comprises manufacturing additional units of the product using the product manufacturing process provided the Probability of Acceptance (Pa) value meets or crosses a threshold. In an embodiment, the method further comprises designating units of the product manufactured by the product manufacturing process for sale or consumption provided the Probability of Acceptance (Pa) value meets or crosses a threshold.

In an embodiment, the method further comprises determining an expected range for the acceptance criterion for the CQA.

In an embodiment, the acceptance criterion is a single-stage acceptance criterion. In an embodiment, the single-stage acceptance criterion is applied to a single test result for the CQA. In an embodiment, the CQA having a single-stage acceptance criterion applied to a single test result comprises one or more selected from adhesion, assay, bulk density, concentration, disintegration, extractable volume, friability, hardness, integrity, osmolality, osmolarity, particle size distribution, particulate matter, peel, pH, potency, shear, specific gravity, spray pattern, tack, thickness, torque, unit weight, viscosity, blend uniformity, cold flow, droplet size, particle count, sterility assurance level, or weight variation.

In an embodiment, the single-stage acceptance criterion is applied to multiple test results. In an embodiment, the CQA having a single-stage acceptance criterion applied to multiple test results comprises one or more selected from adhesion, assay, bulk density, concentration, disintegration, extractable volume, friability, hardness, integrity, osmolality, osmolarity, particle size distribution, particulate matter, peel, pH, potency, shear, specific gravity, spray pattern, tack, thickness, torque, unit weight, viscosity, blend uniformity, cold flow, droplet size, particle count, sterility assurance level, or weight variation.

In an embodiment, the Probability of Acceptance (Pa) value is, or is derived from, (Pₛᵤ)ⁿ, wherein n=number of units tested and Pₛᵤ=probability that a single unit will pass an acceptance criteria.

In an embodiment, the acceptance criterion comprises, or is derived using, multi-stage acceptance criteria. In an embodiment, the multi-stage acceptance criterion is applied to a single test result. In an embodiment, the CQA having multi-stage acceptance criteria applied to a single test result comprises one or more selected from average weight, cold flow, content uniformity, dissolution, droplet size, drug release, or spray pattern.

In an embodiment, the multi-stage acceptance criterion is applied to multiple test results. In an embodiment, the CQA having multi-stage acceptance criteria applied to multiple test results comprises assay per spray, average weight, cold flow, content uniformity, dissolution, droplet size, drug release, pump metering reproducibility, spray pattern, or weight variation.

In an embodiment, the CQA comprises content uniformity.

In an embodiment, the expected range for the acceptance criterion is determined using:

$${S}_{lo} = s \sqrt{\frac{n - 1}{{X}_{( n - 1 , 1 - \frac{a}{2} )}^{2}}}$$

$${S}_{hi} = s \sqrt{\frac{n - 1}{{X}_{( n - 1 , \frac{a}{2} )}^{2}}}$$

wherein
n=the number of units tested
s=sample standard deviation
sₗₒ=lower 95% confidence level of the standard deviation
sₕᵢ=upper 95% confidence level of the standard deviation
χ²=chi-squared distribution
α=probability that units of the product exceed an acceptance value limit.

In an embodiment, the Probability of Acceptance value (Pa) is, or is derived from, α, wherein α is calculated from:

$${X}_{( n - 1 , \frac{a}{2} )}^{2} = \frac{( n - 1 ) {s}^{2}}{{s}_{li m}^{2}}$$

wherein
n=the number of units of the product tested
s=measured standard deviation
sₗᵢₘ=limiting standard deviation
χ²=chi-squared distribution.

In an embodiment, the method further comprises running a statistical simulation of the product manufacturing process and determining a Probability of Acceptance (Pa) value for CQAs with multi-stage acceptance criteria based on data from the simulation. In an embodiment, the statistical simulation comprises a Monte Carlo simulation.

In an embodiment, the method further comprises determining whether the data collected has a normal or non-normal distribution and determining the Probability of Acceptance (Pa) value using data that has a normal distribution. In an embodiment, determining whether the data collected has a normal or non-normal distribution comprises performing a statistical normality test such as an Anderson-Darling normality test.

In an embodiment, the Probability of Acceptance value is used to monitor the collection of raw data. In an embodiment, the Probability of Acceptance value is used to determine the need for additional testing.

In an embodiment, there is provided a method of processing data collected from a product manufacturing process, the method comprising: identifying a critical quality attribute (CQA) of a product manufactured by the process; identifying a plurality of acceptance criteria for the CQA, wherein a first acceptance criterion of the plurality of acceptance criteria is dependent on a second acceptance criterion of the plurality of acceptance criteria; and based on at least the first and second acceptance criteria and on data collected for the CQA of a plurality of units of the product manufactured by the process, determining, by a hardware computer processor, a Probability of Acceptance (Pa) value that specifies the probability that the first acceptance criterion and/or the second acceptance criterion for a future unit of the product will meet or cross, or will not meet or not cross, a threshold defined for the respective first criterion and/or second criterion.

In an embodiment, there is provided a system to assess risk in a product manufacturing process, the system comprising: a hardware computer processor; and computer readable program code, when processed by the processor, configured to: enable identification of a critical quality attribute (CQA) of a product manufactured by the process, enable identification of an acceptance criterion for the CQA for single and/or multiple units of the product manufactured by the process, and based on data collected for the CQA of a plurality of units of the product manufactured by the process, determine a Probability of Acceptance (Pa) value that specifies the probability that the acceptance criterion for a future unit of the product will meet or cross, or will not meet or not cross, a threshold defined for the acceptance criterion.

In an embodiment, there is provided a system to process data collected from a product manufacturing process, the system comprising: a hardware computer processor; and computer readable program code, when processed by the processor, configured to: enable identification of a critical quality attribute (CQA) of a product manufactured by the process, enable identification of a plurality of acceptance criteria for the CQA, wherein a first acceptance criterion of the plurality of acceptance criteria is dependent on a second acceptance criterion of the plurality of acceptance criteria, and determine, based on at least the first and second acceptance criteria and data collected for the CQA of a plurality of units of the product manufactured by the process, a Probability of Acceptance (Pa) value that specifies the probability that the first acceptance criterion and/or the second acceptance criterion for a future unit of the product will meet or cross, or will not meet or not cross, a threshold defined for the respective first criterion and/or second criterion.

Further details can be found in the paper by one or more of the inventors titled D. Alsemeyer et al., “Acceptance Probability (Pa) Analysis for Process Validation Lifecycle Stages”, AAPS PharmSciTech, Vol. 17, No. 2, pages 516-22 (April 2016), which is incorporated herein in its entirety by reference.

The following non-limiting examples and embodiments are provided to further illustrate the present description.

### Probability of Acceptance Analysis

As with traditional capability indices, Probability of Acceptance analysis assumes that underlying data distributions are normal. Non-normal or skewed distributions should be approached with more sophisticated statistical modeling techniques. There is also an implied assumption that maintaining the current manufacturing and raw material controls, future batches shall behave as per the modelled data, which is the fundamental concept for process validation. The following examples illustrate the method for a few select critical quality attributes (CQAs). In an embodiment, the normality of the data is examined, such as using an Anderson Darling Normality Test, before proceeding to further analysis.

### Example: CQA Having One-Stage Acceptance Criteria—Hardness, Thickness, Unit Weight

In-process examinations often assess multiple units that have one-stage acceptance criteria. For example, five tablets may be removed at a regular interval from the process and individually tested for hardness. If any single tested tablet in the examined group does not meet the hardness specification criteria, the examination fails (referred to as “zero acceptance”) and a corrective action is taken on the process. The Probability of Acceptance (Pₐ) for a specific examination is dependent on the number of units tested (n) and the probability that a single unit (Pₛᵤ) will pass the specification criteria.

For a CQA having one-stage acceptance criteria, Probability of Acceptance (Pₐ) can be calculated from the following equation:

Pₐ=(Pₛᵤ)ⁿ

As with previous measures, confidence limits may be assessed that depend on the number of samples acquired.

Similar techniques could be applied to other attributes with a one-stage acceptance criteria.

### Example: CQA Having Two-Stage Acceptance Criteria—Content Uniformity

USP <905> describes a currently accepted methodology to assess the consistency of dosage units within a given batch of product. The uniformity of dosage units can be demonstrated by either of two methods, content uniformity (CU) test or weight variation (WV) test as specified in USP <905>.

The content uniformity test calls for a two-tiered testing approach wherein thirty dosage units are selected. Ten of the dosage units (e.g., tablets) are assayed individually for tier 1 using the appropriate analytical method. If the ten tablets meet the acceptance criteria, then content uniformity has been established. If the tablets in this sample do not meet the acceptance criteria, the remaining 20 dosage units are assayed and are evaluated in combination with the individual assay results from tier 1, using the criteria for tier 2, The sample sizes in tier 1 (L1) and tier 2 (L2) are inflexibly fixed.

The acceptance value, AV, is calculated as according to the formula:

AV=|M−X|+ks

wherein,
X=sample mean as % of label claim (% LC)
k=acceptability constant (wherein k=2.4 for L1 and k=2.0 for L2);
s=sample standard deviation
M=reference value which depends on the sample mean (wherein if 98.5% LC≤X≤101.5% LC, then M=X; if X<98.5% LC, then M=98.5; and if X>101.5% LC, then M=101.5).

The maximum allowed acceptance value is 15.0 at L1 and is 25.0 at L2. Also, in this example, there is an additional requirement that no individual tablet in L2 is outside the range (0.75)(M) to (1.25)(M). Put another way, no unit can be <0.75M and no unit can be >1.25M.

In some cases USP <905> works fine for CU testing, but there can be flaws. One limitation is the fixed samples sizes for L1 and L2, which are 10 and 30, respectively. Since L1 (especially) and L2 are relatively small, the acceptance value calculation may not provide a confident estimation of the batch compared to larger sample sizes. A second limitation is that limits are placed on individual tablets in L2 and they may vary according to the sample mean. Further, the reference value M depends on the sample mean X in such a way that there is a 1.5% zone of indifference around 100% LC.

USP <905> describes testing 10 units at L1 (k=2.4) and 30 units at stage L2 (k 2.0). So, there are three cases each to consider for both the L1 and L2 acceptance value as summarized in Table 3.

TABLE 3
Summary of the three cases per USP <905>

| Case | Mean | L1 | L2 |
|---|---|---|---|
| Case A | 98.5% > x̅ | AV = 98.5 − x̅ + | AV = 98.5 − x̅ + |
| | | 2.4 s | 2.0 s |
| Case B | 98.5% ≤ x̅ ≤ | AV = 2.4 s | AV = 2.0 s |
| | 101.5% | | |
| Case C | x̅ > 101.5% | AV = x̅ − 101.5 + | AV = x̅ − 101.5 + |
| | | 2.4 s | 2.0 s |

As mentioned above, the acceptance value limit for L1 is 15.0 and the acceptance value limit for L2 is 25.0. Using these values, one can work backwards to determine a limiting standard deviation (Sₗᵢₘ) to pass the USP <905> Content Uniformity test for each case.

For Case A, Sₗᵢₘ for passing L1 can be back calculated as:

$$\frac{X - 98.5 + 15}{2.4} = \frac{X}{24 - 34.79}$$

For Case A, Sₗᵢₘ for passing L2 can be calculated as:

$$\frac{X - 98.5 + 25}{2.0} = \frac{X}{2.0 - 36.75}$$

The limiting standard deviations to pass L1 and L2 in Case B and Case C can be calculated in a similar manner. These solutions are provided in Table 4.

TABLE 4
Calculations for Limiting Standard Deviation (Sₗᵢₘ) for Cases A-C

| Case | Mean | L1 | L2 |
|---|---|---|---|
| Case A | 98.5% > x | Sₗᵢₘ = x/2.4 − 34.79 | Sₗᵢₘ = x/2.0 − 36.75 |
| Case B | 98.5% ≤ x ≤ | Sₗᵢₘ = 6.25 | Sₗᵢₘ = 12.5 |
| | 101.5% | | |
| Case C | x > 101.5% | Sₗᵢₘ = 48.54 − x/2.4 | Sₗᵢₘ = 63.25 − x/2.0 |

The lower confidence limit (Sₗₒ) and upper confidence limit (Sₕᵢ) for the standard deviation are described by a chi-square distribution. These limits provide an acceptance value (AV) range for content uniformity.

$${S}_{lo} = s \sqrt{\frac{n - 1}{{X}_{( n - 11 - \frac{a}{2} )}^{2}}}$$

$${s}_{h i} = s \sqrt{\frac{n - 1}{{X}_{( n - {1}_{1} \frac{c ι}{2} )}^{2}}}$$

wherein
n=the number of units tested
s=sample standard deviation
sₗₒ=lower 95% confidence level of the standard deviation
sₕᵢ=upper 95% confidence level of the standard deviation
χ²=chi-squared distribution
α=probability that units of the product exceed an acceptance value limit.

Substituting Sₕᵢ with Sₗᵢₘ and solving for χ² provides:

$${χ}_{( n - {1}_{l} \frac{α}{2} )}^{2} = \frac{( n - 1 ) {s}^{2}}{{s}_{li m}^{P}}$$

Thus, one can assess a, the probability that a test will cross (e.g., exceed) the acceptance value (AV) limit for each case from the chi-square distribution and the measured dosage uniformity data. Specifically, the probability can be back-calculated via the probability density function (pdf) of the chi-square distribution that can be commonly obtained from calculation tools that incorporate statistical calculation components such as Excel.

In an embodiment, the final Probability of Acceptance (Pₐ) value is equivalent to (1-α) for all CQA with single stage or multiple stage acceptance criteria, where α is the probability of not meeting the single stage or multi stage acceptance criteria. This Pₐ value provides an estimate of the chance that a future batch will meet the acceptance value requirements as long as the entered statistics remain descriptive of the process population (i.e. the process remains consistent and stable as observable results). As will be appreciated, the Probability of Acceptance (Pₐ) value can be other forms derived from α.

Thus, the Probability of Acceptance analysis for USP acceptance criteria demonstrates how future product risk can be measured on a consistent and readily understandable basis. The Probability of Acceptance analysis can provide an understanding of the product probability of meeting both tier 1 and tier 2 criteria. Since USP <905> allows for analyzing a reduced number of tablets if tier 1 criteria are met, the Pₐ determination will help in making an informed decision as to whether to go directly with testing 30 dosage units or start with testing 10 units. This will potentially help to eliminate excessive sample testing cycles in Stage 1, Stage 2, and/or Stage 3 of process validation.

Similar techniques could be applied to other attributes with two-stage acceptance criteria.

### Example: CQA Having Three-Stage Acceptance Criteria—Dissolution

As in dosage uniformity, dissolution testing uses multiple tested units and follows a stage-wise acceptance criterion. Because of this, traditional process capability (Cpk) indices fall short in providing a reliable assessment of the ability of the product to meet the acceptance criteria.

Acceptance criteria for dissolution testing follows rules that are outlined in USP General Chapter <711> Dissolution [The United States Pharmacopeia Convention, General Chapter <711> Dissolution, The United States Pharmacopeia and The National Formulary, Dec. 1, 2011 (“USP <711>”), incorporated herein by reference in its entirety]. USP <711> provides information about conditions and procedures of the tests and acceptance criteria which the drug must meet to be accepted. There are separate acceptance tables for immediate release (IR) dosage forms, extended release (ER) dosage forms, and delayed release (DR) dosage forms.

The dissolution test is performed as instructed in USP <711>. The requirements are met if the quantities of active ingredient dissolved from the tested units conform to the acceptance tables for that dosage form. Testing is continued through three stages unless the results conform at stage S1 or S2. The quantity, Q, is the amount of dissolved active ingredient specified in USP <711>, expressed in percentage label claim (% LC). The 5%, 15%, and 25% values in the acceptance table are expressed in % LC so that these values and Q are in the same terms.

The USP rules for immediate release dosage forms are shown in Table 5.

TABLE 5
USP Rules for Immediate Release Dosage Forms

| | Number | |
|---|---|---|
| Stage | of Units | Acceptance Criteria |
| 1 | 6 | Each unit is not less than Q + 5% |
| 2 | 6 | Average of 12 units (S1 + S2) is ≥ Q |
| | | and no unit is < Q − 15% |
| 3 | 12 | Average of 24 units (S1 + S2 + S3) is Q, |
| | | not more than two units < Q − 15%, and no |
| | | unit is < Q − 25% |

These acceptance criteria rules are inter-related and become quite complicated to solve analytically. However, in an embodiment, the probability of meeting the acceptance criteria (Pₐ) for a future batch at a particular stage can be estimated by comparing the pooled dissolution statistics (average and standard deviation) of the measured batches against data derived from a Monte Carlo simulation of the USP acceptance criteria guidelines with defined batch averages and variability.

Conceptually, a Monte Carlo simulation is a broad class of computational algorithms that use repeated random sampling to obtain the distribution of an unknown probabilistic entity. Monte Carlo simulation is often useful when it is challenging to obtain a closed-form expression such as the probability of passing a multi-stage testing. Its simulation can provide a “virtual” manufacturing plant that “produces units” that are defined by a normal distribution of dissolution results with a mean and standard deviation described by the historical batch sample results.

So, in an embodiment, a multitude of “batches” are “manufactured” and selected “manufactured units” are “tested” as per USP <711> criteria. The number of batches that pass a given stage criteria are compared to the total number of batches “produced” to provide the probability that a batch with defined normal distribution characteristics will be accepted. The “virtual plant” is then reset with a new set of normal distribution characteristics and the process repeated.

An example flow chart for a Monte Carlo simulation for the USP <711> stage-wise acceptance criteria for immediate release dissolution testing is shown in FIG. 14. In this process, a simulated batch is initiated and tablets are generated with normal distribution characteristics based on the measured analytical results from the validation batches. The USP criteria are applied to the simulated tablets to assess if the specific batch meets the USP criteria. The number of tablets that pass a given stage criteria are compared to the total number of tablets produced to provide the probability that a batch with the defined normal distribution characteristics will be accepted.

For instance, the Stage 1 (S1) criterion is passed if all six units tested are ≥Q+5%. This probability can be calculated as:

P{Pass S1}=p⁶

wherein p is the probability of a single unit result is greater than Q+5%.

For the samples that fail S1, a new sample of 6 is then generated, combined with the first 6 units and tested against Stage 2 (S2) criteria. Similarly, more units can be generated accordingly to test against Stage 3 (S3) criteria.

Lower 95% confidence limits are generated by repeating the assessment with the lower 95% confidence level of the mean and the upper 95% confidence level of the standard deviation. This provides the lower bound to the acceptance probability determination.

This simulation process may be altered with a new set of normal distribution characteristics and the process repeated to generate tables of Probability of Acceptance (Pₐ) for each characteristic such as hardness, thickness, and/or tablet weight for solid dosage forms. The process is run through different combinations of parameters—Q, mean, standard deviation (STD), and so forth—selected in a Monte Carlo fashion.

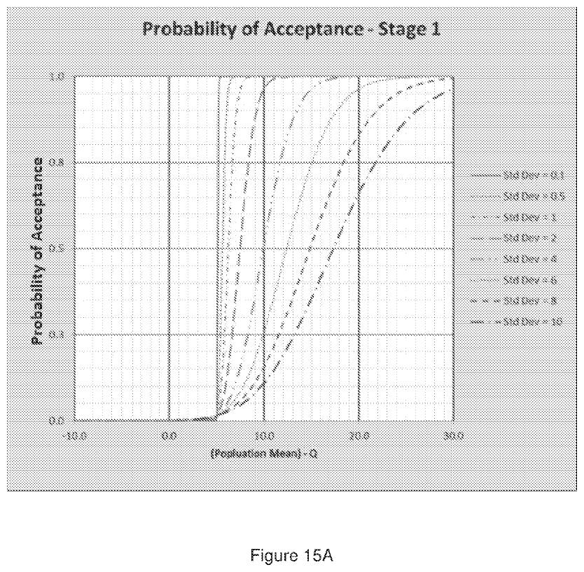

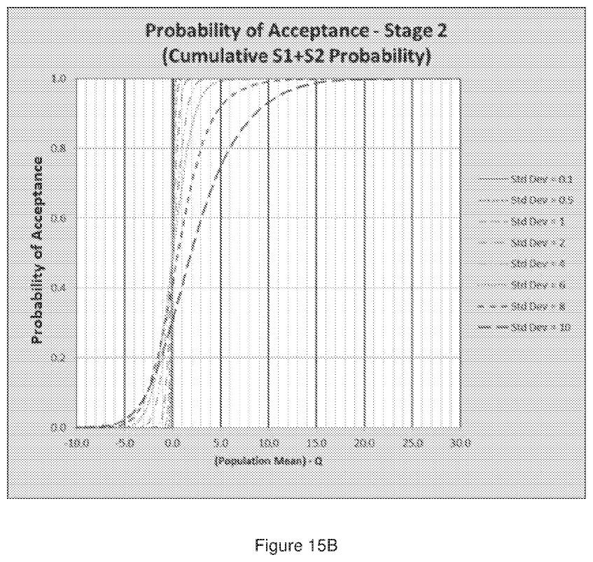

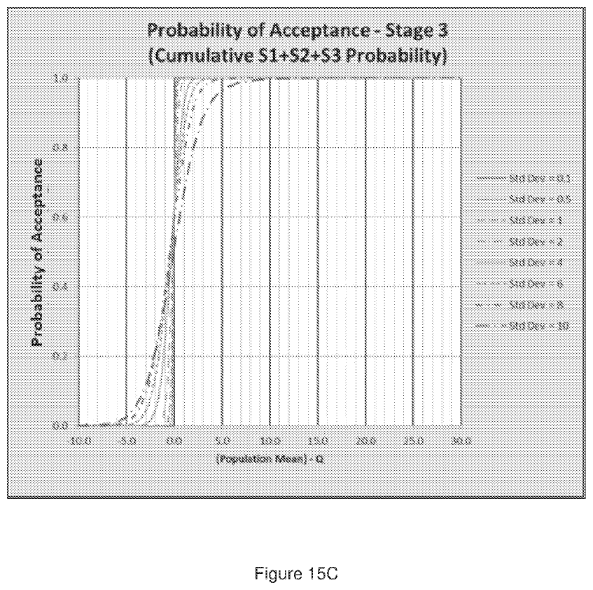

Charts or “operating curves” are developed from the tabular data that provide the Probability of Acceptance (Pₐ) for various virtual plant population means and standard deviations. These charts plot the Probability of Acceptance (Pₐ) of (immediate release) dissolution as a function of percent of individual results greater than Q. FIGS. 15A, 15B, and 15C depict example Probability of Acceptance charts for immediate release dissolution tests at Stage 1, Stage 2, and Stage 3, respectively.

Rather than deriving analytical equations that estimate the probability, a Monte Carlo simulation generates these “operating curves” that provide a solution to the complex stage-wise equation. More simulation iterations provide a higher precision to the solution.

To assess Probability of Acceptance (Pₐ), the computed mean and standard deviation of the validation batches are located on the charts. For example, if the dissolution results from the campaign for a product are 90% (the amount of dissolved active ingredient expressed in percentage label claim (% LC)) with a standard deviation of 4% and the dissolution acceptance criterion is Q=80% at the specified Q time, then the Stage 1 Probability of Acceptance is about 0.50. That is, there is a 50% chance that a future batch of product will pass the S1 criterion.

With a 90% average dissolution result and standard deviation of 4%, there is >99.99% probability that a future product batch will meet the S2 acceptance criteria with a Q=80% at the specified Q time, And, there is >99.99% probability that a future product batch will meet the S3 criteria as well. Confidence limits are used to indicate how well the determined dissolution capability is known. The probability of meeting each particular stage acceptance criteria and the associated lower 95% confidence limits can be determined for each stage by using the “worst-case” intervals for both the mean and standard deviation to perform the Monte Carlo simulation.

Another example flow chart for a Monte Carlo simulation for the USP <711> stage-wise acceptance criteria for immediate release dissolution testing is shown in FIG. 16.

Using the principles described herein in relation to the specific process parameter acceptance criteria, along with the fundamental equations and confidence interval statistics, one can adapt the techniques herein to apply the Probability of Acceptance concept to any quality attribute. So, similar techniques as described could be applied to other attributes with a three or more-stage acceptance criteria.

Also, the concept is readily applicable to sterile/non sterile liquid dose products, Quality attributes such as deliverable volume and assay per spray have stage-wise acceptance that can be converted into a Probability of Acceptance.

### Example: Correlation Between Capability Indices and Probability of Acceptance

Process capability (Cpk) indices were developed as part of traditional statistical process control strategies to denote the ability of a process to produce output within specifications limits. Table 6 provides a correlation between Cpk values, the sigma level (i.e., the number of sigma between the process mean and the nearest specification limit), the anticipated Probability of Acceptance, and the expected level of fraction defects. The Probability of Acceptance and corresponding fraction defects are determined for Table 6 using the sigma level and a normal distribution with a 1.5 sigma shift, which is an industry practice.

TABLE 6
Correlation between Process Capability (Cpk), Sigma
Level, Probability of Acceptance (Pa), and Fraction Defects

| Process | | Probability of | |
|---|---|---|---|
| Capability | Sigma | Acceptance | Fraction |
| (Cₚₖ) | Level | (Pₐ) | Defects |
| 0.33 | 1 | 30.8537533% | 691,462 |
| 0.67 | 2 | 69.1462467% | 308,537 |
| 1.00 | 3 | 99.3192771% | 66,807 |
| 1.33 | 4 | 99.3790320% | 6210 |
| 1.67 | 5 | 99.9767327% | 233 |
| 2.00 | 6 | 99.9996599% | 3.4 |

### Process and Product Lifecycle

In an embodiment, the critical quality attribute (CQA) comprises one or more selected from adhesion, assay (per spray), blend uniformity, bulk density, cold flow, concentration, content uniformity, dissolution, disintegration, droplet size, drug release, fill weight, extractable volume, friability, hardness, integrity, osmolality, osmolarity, particle size distribution, particle count, particulate matter, peel, pH, potency, shear, specific gravity, spray pattern, sterility assurance level, tack, thickness, torque, unit weight, viscosity, or weight variation.

In an embodiment, the product described herein comprises a parenteral, solid oral, liquid, transdermal, or dry powder inhalation formulation for animal or human use.

In an embodiment, the product described herein comprises an active pharmaceutical ingredient, a human drug product, a biological product, a veterinary drug product, a dietary supplement, a nutraceutical, a cosmetic, a radiopharmaceutical, an excipient, a medical device, or any combination thereof.

In an embodiment, the product described herein comprises a biological product selected from an antibody, a cytokine, a growth factor, an enzyme, an immunomodulator, a vaccine, a human tissue or tissue product, an allergen, or a blood component.

In an embodiment, the critical quality attribute (CQA) of the biological product comprises one or more selected from concentration, extractable volume, ion exchange chromatography, osmolality, particulate matter, pH, potency, reverse phase chromatography, or size exclusion chromatography.

In an embodiment, the manufacturing process described herein is a batch process, a semi-batch process, a continuous process, a semi-continuous process, or a hybrid batch/continuous process.

In an embodiment, the product described herein is an in-process material or a finished product.

In an embodiment, the product manufacturing process is a commercial process or a development process.

In an embodiment, the product manufacturing process is a pharmaceutical product manufacturing process.

In an embodiment, there is provided a product manufactured based on a method as described herein. In an embodiment, there is provided a product assessed using a method as described herein.

In an embodiment, the product comprises an active pharmaceutical ingredient (API), a human drug product, a biological product, a veterinary drug product, a dietary supplement, a nutraceutical, a cosmetic, a radiopharmaceutical, an excipient, .a medical device, or any combination thereof. In an embodiment, the product is an in-process material or a finished product. In an embodiment, the product is a commercial product or a development product. In an embodiment, the product is a commercial product or a development product undergoing a change of continuous improvement.

In an embodiment, modifying the product manufacturing process comprises changing an apparatus used in the process, changing a process parameter (e.g., temperature, force applied, speed, etc.) of the process, changing a setting of an apparatus used in the process, changing a type, or a materials, chemical, dimensional, temperature, etc. parameter, of an ingredient of the products manufactured using the process, etc.

### Embodiments

Embodiments of the invention are described in the following clauses:

1. A method for risk assessment in a product manufacturing process, the method comprising: identifying a critical quality attribute (CQA) of a product manufactured by the process; identifying an acceptance criterion for the CQA for single and/or multiple units of the product manufactured by the process; and based on data collected for the CQA of a plurality of units of the product manufactured by the process, determining, by a hardware computer processor, a Probability of Acceptance (Pa) value that specifies the probability that the acceptance criterion for a future unit of the product will meet or cross, or will not meet or not cross, a threshold defined for the acceptance criterion.

2. The method of clause 1, further comprising modifying the product manufacturing process based on the Probability of Acceptance (Pa) value.

3. The method of clause 2, further comprising modifying equipment or a test method.

4. The method of clause 2, further comprising modifying the product manufacturing process at Process Validation Stage 1, Stage 2, Stage 3, or any combination thereof.

5. The method of any of clauses 1-4, further comprising manufacturing additional units of the product using the product manufacturing process provided the Probability of Acceptance (Pa) value meets or crosses a threshold.

6. The method of any of clauses 1-5, further comprising designating units of the product manufactured by the product manufacturing process for sale or consumption provided the Probability of Acceptance (Pa) value meets or crosses a threshold.

7. The method of any of clauses 1-6, further comprising determining an expected range for the acceptance criterion for the CQA.

8. The method of any of clauses 1-7, wherein the acceptance criterion is a single-stage acceptance criterion for the CQA.

9. The method of clause 8, wherein a single-stage acceptance criterion is applied to a single test result for the CQA.

10. The method of clause 9, wherein the CQA comprises one or more selected from adhesion, assay, bulk density, concentration, disintegration, extractable volume, friability, hardness, integrity, osmolality, osmolarity, particle size distribution, particulate matter, peel, pH, potency, shear, specific gravity, spray pattern, tack, thickness, torque, unit weight, viscosity, blend uniformity, cold flow, droplet size, particle count, sterility assurance level, or weight variation.

11. The method of clause 8, wherein a single-stage acceptance criterion is applied to multiple test results.

12. The method of clause 11, wherein the CQA comprises one or more selected from adhesion, assay, bulk density, concentration, disintegration, extractable volume, friability, hardness, integrity, osmolality, osmolarity, particle size distribution, particulate matter, peel, pH, potency, shear, specific gravity, spray pattern, tack, thickness, torque, unit weight, viscosity, blend uniformity, cold flow, droplet size, particle count, sterility assurance level, or weight variation.

13. The method of clauses 11 or 12, wherein the Probability of Acceptance (Pa) value is, or is derived from, (Pₛᵤ)ⁿ wherein: n=number of units tested and Pₛᵤ=probability that a single unit will pass an acceptance criteria.

14. The method of any of clauses 1-7, wherein the acceptance criterion comprises, or is derived using, multi-stage acceptance criteria.

15. The method of clause 14, wherein the multi-stage acceptance criterion is applied to a single test result.

16. The method of clause 15, wherein the CQA comprises one or more selected from average weight, cold flow, content uniformity, dissolution, droplet size, drug release, or spray pattern.

17. The method of clause 14, wherein the multi-stage acceptance criterion is applied to multiple test results.

18. The method of clause 17, wherein the CQA comprises one or more selected from assay per spray, average weight, cold flow, content uniformity, dissolution, droplet size, drug release, pump metering reproducibility, spray pattern, or weight variation.

19. The method of any of clauses 14-18, wherein the CQA comprises content uniformity.

20. The method of any of clauses 14-19, wherein the expected range for the acceptance criterion is determined using:

$${S}_{lo} = s \sqrt{\frac{n - 1}{{X}_{( n - 1 , 1 - \frac{a}{2} )}^{2}}}$$

$${S}_{hi} = s \sqrt{\frac{n - 1}{{X}_{( n - 1 \frac{a}{2} )}^{2}}}$$

wherein: n=the number of units tested, s=sample standard deviation, sₗₒ=lower 95% confidence level of the standard deviation, sₕᵢ=upper 95% confidence level of the standard deviation, χ²=chi-squared distribution, and a=probability that units of the product exceed an acceptance value limit.

21. The method of any of clauses 14-20, wherein the Probability of Acceptance value (Pa) is, or is derived from, α, wherein α is calculated from:

$${X}_{( n - 1 , \frac{a}{2} )}^{2} = \frac{( n - 1 ) {s}^{2}}{{s}_{li m}^{2}}$$

wherein n=the number of units of the product tested, s=measured standard deviation, sₗᵢₘ=limiting standard deviation, and χ²=chi-squared distribution.

22. The method of any of clauses 14-21, further comprising running a statistical simulation of the product manufacturing process and determining a Probability of Acceptance (Pa) value for a CQA with the multi-stage acceptance criterion based on data from the simulation.

23. The method of clause 22, wherein the statistical simulation comprises a Monte Carlo simulation.

24. The method of any of clauses 1-23, further comprising determining whether the data collected has a normal or non-normal distribution, and determining the Probability of Acceptance (Pa) value using data that has a normal distribution.

25. The method of clause 24, wherein determining whether the data collected has a normal or non-normal distribution comprises performing a statistical normality test such as an Anderson-Darling normality test.

26. The method of any of clauses 1-25, wherein the critical quality attribute (CQA) comprises one or more selected from adhesion, assay (per spray), blend uniformity, bulk density, cold flow, concentration, content uniformity, dissolution, disintegration, droplet size, drug release, fill weight, extractable volume, friability, hardness, integrity, osmolality, osmolarity, particle size distribution, particle count, particulate matter, peel, pH, potency, shear, specific gravity, spray pattern, sterility assurance level, tack, thickness, torque, unit weight, viscosity, or weight variation.

27. The method of any of clauses 1-26, wherein the product is a parenteral, solid oral, liquid, transdermal, or dry powder inhalation formulation, for animal or human use.

28. The method of any of clauses 1-27, wherein the product comprises an active pharmaceutical ingredient, a human drug product, a biological product, a veterinary drug product, a dietary supplement, a nutraceutical, a cosmetic, a radiopharmaceutical, an excipient, a medical device, or any combination thereof.

29. The method of any of clauses 1-27, wherein the product comprises a biological product selected from an antibody, a cytokine, a growth factor, an enzyme, an immunomodulator, a vaccine, a human tissue or tissue product, an allergen, or a blood component.

30. The method of clause 29, wherein the critical quality attribute (CQA) of the biological product comprises one or more selected from concentration, extractable volume, ion exchange chromatography, osmolality, particulate matter, pH, potency, reverse phase chromatography, or size exclusion chromatography.

31. The method of any of clauses 1-30, wherein the manufacturing process is a batch process, a semi-batch process, a continuous process, a semi-continuous process, or a hybrid batch/continuous process.

32. The method of any of clauses 1-31, wherein the product is an in-process material or a finished product.

33. The method of any of clauses 1-32, wherein the product manufacturing process is a commercial process or a development process.

34. The method of any of clauses 1-33, wherein the Probability of Acceptance (Pa) value is used to monitor the collection of raw data.

35. The method of any of clauses 1-34, wherein the Probability of Acceptance (Pa) value is used to determine the need for additional testing.

36. A method of processing data collected from a product manufacturing process, the method comprising: identifying a critical quality attribute (CQA) of a product manufactured by the process; identifying a plurality of acceptance criteria for the CQA, wherein a first acceptance criterion of the plurality of acceptance criteria is dependent on a second acceptance criterion of the plurality of acceptance criteria; and based on at least the first and second acceptance criteria and on data collected for the CQA of a plurality of units of the product manufactured by the process, determining, by a hardware computer processor, a Probability of Acceptance (Pa) value that specifies the probability that the first acceptance criterion and/or the second acceptance criterion for a future unit of the product will meet or cross, or will not meet or not cross, a threshold defined for the respective first criterion and/or second criterion.

37. A computer program product comprising a non-transitory computer readable storage medium comprising computer-readable program code embodied therewith, the computer readable program code comprising computer readable program code configured to perform the method of any of clauses 1-36.

38. A product assessed using the method of any of clauses 1 to 36.

39. The product of clause 38, wherein the product comprises an active pharmaceutical ingredient, a human drug product, a biological product, a veterinary drug product, a dietary supplement, a nutraceutical, a cosmetic, a radiopharmaceutical, an excipient, a medical device, or any combination thereof.

40. The product of clauses 38 or 39, wherein the product is an in-process material or a finished product.

41. The product of any of clauses 38-40, wherein the product is a commercial product or a development product.

42. The product of clause 41, wherein the product is a commercial product or a development product undergoing a change of continuous improvement.

43. A system to assess risk in a product manufacturing process, the system comprising: a hardware computer processor; and computer readable program code, when processed by the processor, configured to: enable identification of a critical quality attribute (CQA) of a product manufactured by the process; enable identification of an acceptance criterion for the CQA for single and/or multiple units of the product manufactured by the process; and based on data collected for the CQA of a plurality of units of the product manufactured by the process, determine a Probability of Acceptance (Pa) value that specifies the probability that the acceptance criterion for a future unit of the product will meet or cross, or will not meet or not cross, a threshold defined for the acceptance criterion.

44. A system to process data collected from a product manufacturing process, the system comprising: a hardware computer processor; and computer readable program code, when processed by the processor, configured to: enable identification of a critical quality attribute (CQA) of a product manufactured by the process; enable identification of a plurality of acceptance criteria for the CQA, wherein a first acceptance criterion of the plurality of acceptance criteria is dependent on a second acceptance criterion of the plurality of acceptance criteria; and determine, based on at least the first and second acceptance criteria and data collected for the CQA of a plurality of units of the product manufactured by the process, a Probability of Acceptance (Pa) value that specifies the probability that the first acceptance criterion and/or the second acceptance criterion for a future unit of the product will meet or cross, or will not meet or not cross, a threshold defined for the respective first criterion and/or second criterion.

Further embodiments of the invention are described in the following clauses:

1. A method of determining a minimum number of lots needed for performance qualification and/or verification of a product manufacturing process, the method comprising: identifying a critical quality attribute (CQA) of a product manufactured by the process; identifying a specification limit for the CQA; based on data collected for the CQA of a plurality of units of the product manufactured by the process, computing, by a hardware computer processor, one or more confidence bands for a plurality of statistical measures of the data collected; and determining a minimum number of lots required for the one or more confidence bands to fall within the specification limit for the CQA.

2. The method of clause 1, wherein the minimum number of lots is for qualification and the qualification is process performance qualification, product performance qualification, method performance qualification, supplied material qualification, equipment qualification, facility qualification, or utility qualification.

3. The method of clause 1, wherein the minimum number of lots is for verification and the verification is design verification or process verification.

4. The method of any of clauses 1 to 3, wherein the data collected for the CQA is from development lots, lots of a similar process, lots of a similar product, or any combination thereof.

5. The method of clause 4, wherein the data collected for the CQA is from lots of a similar process and the process is similar based on product strength, equipment type, a testing property, or any combination thereof and/or the data collected for the CQA is from lots of a similar product and the product is similar based on product strength, equipment type, a testing property, or any combination thereof.

6. The method of any of clauses 1 to 5, further comprising modifying the number of lots needed to qualify and/or verify a process based on data collected from the product manufacturing process.

7. The method of any of clauses 1 to 6, further comprising manufacturing additional units of the product provided data collected from the product manufacturing process meets an acceptance criterion.

8. The method of any of clauses 1 to 7, further comprising designating units of the product for sale or consumption provided data collected from the minimum number of lots of the product manufacturing process meets an acceptance criterion.

9. The method of any of clauses 1 to 8, wherein determining the minimum number of lots comprises determining the minimum number of lots required for the confidence band to fall within the specification limit for the CQA.

10. The method of any of clauses 1 to 9, wherein the CQA is classified as a label claim CQA which has direct end user impact or a non-label claim CQA which has essentially no direct end user impact.

11. The method of any of clauses 1 to 10, wherein the data collected comprises intra-lot variability data for the CQA.

12. The method of clause 10 or 11, wherein the intra-lot data is collected from lots produced for the purposes of a clinical trial, a governmental agency submission/registration study, a stability study, a process scale-up study, a method validation study, or an equipment capability study.

13. The method of any of clauses 1 to 12, wherein the data collected comprises lot-to lot performance data for the CQA.

14. The method of clause 13, wherein the lot-to-lot performance data is estimated using lot-to-lot data collected from a similar product category where similar is defined as a similar label claim or a similar manufacturing process depending on process knowledge and data analysis.

15. The method of any of clauses 1 to 14, wherein the plurality of statistical measures comprises mean and standard deviation.

16. The method of any of clauses 1 to 15, wherein the confidence band is derived from:

$$μ = \overline{x} \pm {t}_{( \frac{a}{2} , n - 1 )} \frac{s}{\sqrt{n}}$$

$$\text{and/or}$$

$$s \sqrt{\frac{n - 1}{{χ}_{( n - 1 , 1 - \frac{a}{2} )}^{2}}} \leq σ \leq s \sqrt{\frac{n - 1}{{χ}_{( n - 1 \cdot \frac{a}{2} )}^{2}}}$$

wherein μ=underlying population mean, σ=underlying standard deviation, n=the number of lots, x̅=measured mean, s=measured standard deviation, α=probability, t=t-distribution, and χ²=chi-squared distribution.

17. The method of any of clauses 1 to 16, further comprising repeating the computing and determining for each of a plurality of critical quality attributes (CQA) of the product, a minimum number of lots required for a respective confidence band to fall within the specification limit for each CQA.

18. The method of clause 17, further comprising selecting, from a minimum number of lots for each CQA, the highest minimum number as the required number of lots needed for qualification and/or verification of the product manufacturing process.

19. The method of any of clauses 1 to 18, wherein computing the one or more confidence bands uses a formula derived from single-stage acceptance criteria for the CQA.

20. The method of any of clauses 1 to 19, wherein computing the one or more confidence bands uses a formula derived from multi-stage acceptance criteria for the CQA.

21. The method of any of clauses 1 to 20, wherein computing the one or more confidence bands comprises running a statistical simulation of the product manufacturing process.

22. The method of clause 21, wherein the statistical simulation comprises a Monte Carlo simulation.

23. The method of any of clauses 1 to 22, further comprising determining whether the data collected has a normal or non-normal distribution and determining the one or more confidence bands based on data that has a normal distribution.

24. The method of clause 23, wherein determining whether the data collected has a normal or non-normal distribution comprises performing a statistical normality test such as the Anderson-Darling normality test.

25. The method of any of clauses 1 to 24, wherein the critical quality attribute (CQA) comprises one or more selected from adhesion, assay (per spray), blend uniformity, bulk density, cold flow, concentration, content uniformity, dissolution, disintegration, droplet size, drug release, fill weight, extractable volume, friability, hardness, integrity, osmolality, osmolarity, particle size distribution, particle count, particulate matter, peel, pH, potency, shear, specific gravity, spray pattern, sterility assurance level, tack, thickness, torque, unit weight, viscosity, or weight variation.

26. The method of any of clauses 1 to 25, wherein the product is a parenteral, solid oral, liquid, transdermal, or dry powder inhalation formulation, for animal or human use.

27. The method of any of clauses 1 to 26, wherein the product comprises an active pharmaceutical ingredient, a human drug, a biological product, a veterinary drug product, a dietary supplement, a nutraceutical, a cosmetic, a radiopharmaceutical, an excipient, a medical device, or any combination thereof.

28. The method of any of clauses 1 to 27, wherein the product comprises a biological product selected from an antibody, a cytokine, a growth factor, an enzyme, an immunomodulator, a vaccine, a human tissue or tissue product, an allergen, or a blood component.

29. The method of clause 28, wherein the critical quality attribute (CQA) of the biological product comprises one or more selected from concentration, extractable volume, ion exchange chromatography, osmolality, particulate matter, pH, potency, reverse phase chromatography, or size exclusion chromatography.

30. The method of any of clauses 1 to 29, wherein the product manufacturing process is a batch process, a semi-batch process, a continuous process, a semi-continuous process, or a hybrid batch/continuous process.

31, The method of any of clauses 1 to 30, wherein the product is an in-process material or a finished product.

32. The method of any of clauses 1 to 31, wherein the product manufacturing process is a commercial process or a development process.

33. The method of any of clauses 1 to 32, wherein the method is used to monitor the collection of raw data.

34. The method of any of clauses 1 to 33, wherein the method is used to determine the need for additional testing.

35. The method of any of clauses 1 to 34, further comprising modifying the product manufacturing process based on data collected from a minimum number of lots of the product manufacturing process.

36. The method of any of clauses 1 to 35, further comprising manufacturing additional units of the product provided data collected from a minimum number of lots of the product manufacturing process meets an acceptance criterion.

37. A method of processing data collected from a product manufacturing process, the data relating to a critical quality attribute (CQA) of a product manufactured by the process, the method comprising: computing, based on data collected, a series of confidence bands, each confidence band being for a different number of lots of the product manufactured by the process; determining whether each confidence band falls within a specification limit for the CQA; selecting a minimum number of lots required for the respective confidence band to fall within the specification limit for the CQA; and selecting from a minimum numbers of lots the highest minimum number as the required number of lots needed for qualification and/or verification of the product manufacturing process.

38. A computer program product comprising a non-transitory computer readable storage medium comprising computer-readable program code embodied therewith, the computer readable program code comprising computer readable program code configured to perform the method of any of clauses 1 to 37.

39. A product manufactured based on the method of any of clauses 1 to 37.

40. The product of clause 39, wherein the product comprises an active pharmaceutical ingredient, a human drug, a biological product, or a veterinary drug, a dietary supplement, a nutraceutical, a cosmetic, a radiopharmaceutical, an excipient, a medical device, or any combination thereof.

41. The product of clauses 39 or 40, wherein the product is an in-process material or a finished product.

42. The product of any of clauses 39 to 41, wherein the product is a commercial product or a development product.

43. The product of clause 42, wherein the product is a commercial product or a development product undergoing a change of continuous improvement.

44. A system to determine a minimum number of lots needed for performance qualification and/or verification of a product manufacturing process, the system comprising: a hardware computer processor; and computer readable program code, when processed by the processor, configured to: enable identification of a critical quality attribute (CQA) of a product manufactured by the process; enable identification of a specification limit for the CQA; and based on data collected for the CQA of a plurality of units of the product manufactured by the process, compute one or more confidence bands for a plurality of statistical measures of the data collected, and determine a minimum number of lots required for the confidence band to fall within the specification limit for the CQA.

45. A system to process data collected from a product manufacturing process, relating to a critical quality attribute (CQA) of a product, the system comprising: a hardware computer processor; and computer readable program code, when processed by the processor, configured to: compute, based on data collected, a series of confidence bands, each confidence band being for a different number of lots of the product manufactured by the process; determine whether each confidence band falls within a specification limit for the CQA; select a minimum number of lots required for the respective confidence band to fall within the specification limit for the CQA; and select from a minimum numbers of lots the highest minimum number as the required number of lots needed for qualification and/or verification of the product manufacturing process.

Further embodiments of the invention are described in the following clauses:

1. A method for analysis of a product manufacturing process, the method comprising: identifying a critical quality attribute (CQA) of a product manufactured by the process; identifying a plurality of material attributes, a plurality of process parameters, or both, for the CQA, wherein each material attribute and/or process parameter has a risk attribute for the critical quality attribute based on data; classifying each of the material attributes and/or process parameters, based on the associated risk attribute of that material attribute and/or process parameter, for the CQA into a category of a plurality of categories, each category having an assigned risk; and determining, by a hardware computer processor, a risk ratio for the critical quality attribute (CQA) based on the classification of the material attributes and/or process parameters into the categories.

2. The method of clause 1, wherein the critical quality attribute (CQA) is classified as a label claim CQA which has direct end user impact or a non-label claim CQA which has essentially no direct end user impact.

3 The method of clause 2, wherein the critical quality attribute (CQA) is classified as a label claim CQA which has direct end user impact.

4. The method of clause 3, wherein the label claim CQA comprises assay, concentration, content uniformity, dissolution, drug release, fill weight, or potency.

5. The method of clause 2, wherein the critical quality attribute (CQA) is classified as a non-label claim CQA which has essentially no direct end user impact.

6. The method of clause 5, wherein the non-label claim CQA comprises adhesion, assay per spray, blend uniformity, bulk density, cold flow, disintegration, droplet size, extractable volume, friability, hardness, integrity, osmolality, osmolarity, particle count, particle size distribution, particulate matter, peel, pH, shear, specific gravity, spray pattern, sterility assurance level, tack, thickness, torque, unit weight, viscosity, or weight variation.

7. The method of any of clauses 1-6, wherein the risk ratio is based on a count of each of the product material attributes and/or processing parameters in each of the categories.

8, The method of any of clauses 1-7, wherein the risk attributes comprise a risk attribute of whether the material attribute and/or process parameter is critical or not critical to the product, a risk attribute of whether the material attribute and/or process parameter has been evaluated or not evaluated, and a risk attribute of whether the material attribute and/or process parameter has been mitigated or not mitigated.

9. The method of any of clauses 1-7, wherein the categories include a first category for a material attribute and/or process parameter with a critical risk attribute and a not evaluated risk attribute, a second category for a material attribute and/or process parameter with a critical risk attribute and a not mitigated risk attribute, and a third category for a material attribute and/or process parameter with a critical risk attribute and a mitigated risk attribute.

10. The method of 9, wherein the risk ratio is a sum of the count of material attributes and/or process parameters in the first and second categories divided by a sum of the count of material attributes and/or process parameters in the first, second and third categories.

11. The method of any of clauses 1-10, wherein each of the categories is assigned a risk factor, at least two of the risk factors being different.

12. The method of clause 11, wherein the categories are for a non-label claim CQA and two or more of the risk factors are different integers selected from the group consisting of 0, 1, 2, 3, 4, and 5.

13. The method of clause 11, wherein the categories are for a label claim CQA and two or more of the risk factors are different integers selected from the group consisting of 0, 2, 4, 6, 8, and 10.

14. The method of any of clauses 11-13, wherein a risk score is based on a count of each of the material attributes and/or process parameters per category multiplied by the risk factor for that category.

15. The method of any of clauses 1-14, further comprising applying risk attributes to the material attributes and/or process parameters based on a decision tree.

16. The method of clause 15, wherein the decision tree comprises applying to a material attribute and/or a process parameter a risk attribute of whether the material attribute and/or process parameter is critical or not critical to the product, applying to the material attribute and/or process parameter a risk attribute of whether the material attribute and/or process parameter has been evaluated or not evaluated provided it has a critical or not critical risk attribute, and applying to the material attribute and/or the process parameter a risk attribute of whether the material attribute and/or process parameter has been mitigated or not mitigated provided it has an evaluated or not evaluated risk attribute.

17. The method of any of clauses 1-16, further comprising generating, by a hardware computer processor, a risk assessment chart for the critical quality attribute (CQA), the chart providing an indication of the category, for the CQA, for one or more of the material attributes and/or process parameters.

18. The method of clause 17, wherein the risk assessment chart comprises an area chart, a bar chart, a bubble chart, a cone chart, a doughnut chart, a line chart, a Pareto chart, a pie chart, a radar chart, a scatter chart, a surface chart, a heat map, or a prioritization matrix.

19. The method of any of clauses 1-18, further comprising identifying at least one material attribute or process parameter from the plurality of material attributes and process parameters for optimization.

20. The method of any of clauses 1-19, further comprising developing a control strategy for the product manufacturing process based on the risk ratio.

21. The method of any of clauses 1-20, further comprising modifying the product manufacturing process based on the risk ratio.

22. The method of any of clauses 1-21, wherein the critical quality attribute (CQA) is identified from a formulation process, a product development process, a product optimization process, or any combination thereof.

23, The method of any of clauses 1-22, wherein the material attributes and/or process parameters are identified from a formulation process, a product development process, a product optimization process, or any combination thereof.

24. The method of any of clauses 1-23, comprising identifying the plurality of material attributes and wherein one or more of the material attributes are of raw material.

25. The method of any of clauses 1-24, comprising identifying the plurality of process parameters and wherein one or more of the process parameters impact a critical quality attribute.

26. The method of any of clauses 1-25, comprising determining the risk ratio for a formulation, a product development process, a product optimization process, or any combination thereof.

27. The method of any of clauses 1-25, comprising determining the risk ratio for a commercial process.

28. The method of any of clauses 1-27, wherein the critical quality attribute (CQA) comprises one or more selected from adhesion, assay (per spray), blend uniformity, bulk density, cold flow, concentration, content uniformity, dissolution, disintegration, droplet size, drug release, fill weight, extractable volume, friability, hardness, integrity, osmolality, osmolarity, particle size distribution, particle count, particulate matter, peel, pH, potency, shear, specific gravity, spray pattern, sterility assurance level, tack, thickness, torque, unit weight, viscosity, or weight variation.

29. The method of any of clauses 1-28, wherein the product is a parenteral, solid oral, liquid, transdermal, or dry powder inhalation formulation, for animal or human use.

30. The method of any of clauses 1-29, wherein the product comprises an active pharmaceutical ingredient, a human drug, a biological product, a veterinary drug, a dietary supplement, a nutraceutical, a cosmetic, a radiopharmaceutical, an excipient, a medical device, or any combination thereof.

31. The method of any of clauses 1-29, wherein the product comprises a biological product selected from an antibody, a cytokine, a growth factor, an enzyme, an immunomodulator, a vaccine, a human tissue or tissue product, an allergen, or a blood component.

32. The method of clause 31, wherein the critical quality attribute of the biological product comprises one or more selected from concentration, extractable volume, ion exchange chromatography, osmolality, particulate matter, pH, potency, reverse phase chromatography, or size exclusion chromatography.

33. The method of any of clauses 1-32, wherein the manufacturing process is a batch process, a semi-batch process, a continuous process, a semi-continuous process, or a hybrid batch/continuous process.

34. The method of any of clauses 1-33, wherein the product is an in-process material or a finished product.

35. The method of any of clauses 1-34, wherein the product manufacturing process is a commercial process or a development process.

36. The method of any of clauses 1-35, wherein the risk ratio for the critical quality attribute (CQA) is used to monitor the collection of raw data.

37. The method of any of clauses 1-36, wherein the risk ratio for the critical quality attribute (CQA) is used to determine the need for additional testing and/or need for designing an additional study.

38. A computer program product comprising a non-transitory computer readable storage medium comprising computer-readable program code embodied therewith, the computer readable program code comprising computer readable program code configured to perform the method of any of clauses 1-37.

39. A product manufactured based on the method of any of clauses 1-37.

40. The product of clause 39, wherein the product comprises an active pharmaceutical ingredient, a human drug, a biological product, a veterinary drug, a dietary supplement, a nutraceutical, a cosmetic, a radiopharmaceutical, an excipient, a medical device, or any combination thereof.

41. The product of clauses 39 or 40, wherein the product is an in-process material or a finished product.

42. The product of any of clauses 39-41, wherein the product is a commercial product or a development product.

43. The product of clause 42, wherein the product is a commercial product or a development product undergoing a change of continuous improvement.

44. A system to analyze a product manufacturing process, the system comprising: a hardware computer processor; and computer readable program code, when processed by the processor, configured to: enable identification of a critical quality attribute (CQA) of a product manufactured by the process; enable identification of a plurality of material attributes, a plurality of process parameters of the process, or both, for the CQA, wherein each material attribute and/or process parameter has a risk attribute for the critical quality attribute based on data; classify each of the material attributes and/or process parameters, based on the associated risk attribute of that material attribute and/or process parameter, for the CQA into a category of a plurality of categories, each category having an assigned risk; and determine a risk ratio for the critical quality attribute (CQA) based on the classification of the material attributes and/or process parameters into the categories.

### Computer Programs and Systems

In an embodiment, there is provided a computer program product comprising a non-transitory computer readable storage medium comprising computer-readable program code embodied therewith, the computer readable program code comprising computer readable program code configured to perform a method as described herein,

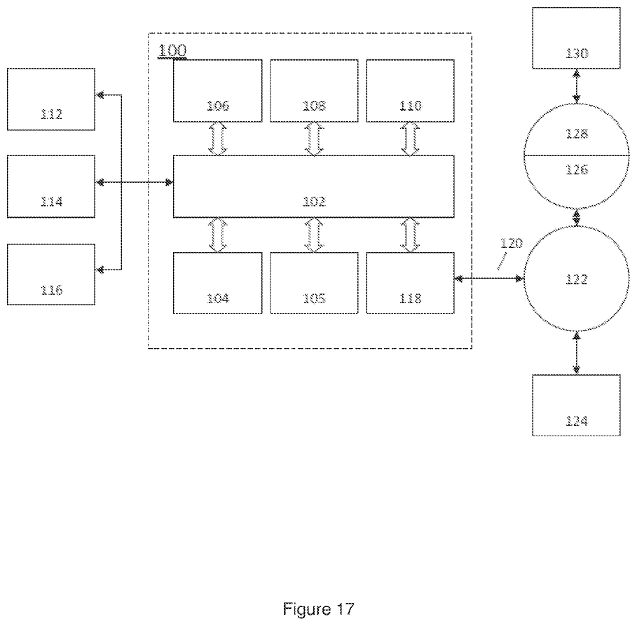

FIG. 17 is a block diagram that illustrates a computer system 100 which can assist in implementing the methods and flows disclosed herein. Computer system 100 includes a bus 102 or other communication mechanism for communicating information, and a processor 104 (or multiple processors 104 and 105) coupled with bus 102 for processing information. Computer system 100 also includes a main memory 106, such as a random access memory (RAM) or other dynamic storage device, coupled to bus 102 for storing information and instructions to be executed by processor 104. Main memory 106 also may be used for storing temporary variables or other intermediate information during execution of instructions to be executed by processor 104. In an embodiment, computer system 100 further includes a read only memory (ROM) 108 or other static storage device coupled to bus 102 for storing static information and instructions for processor 104. In an embodiment, a storage device 110, such as a magnetic disk or optical disk, is provided and coupled to bus 102 for storing information and instructions.

In an embodiment, computer system 100 is coupled via bus 102 to a display 112, such as a cathode ray tube (CRT) or flat panel or touch panel display for displaying information to a computer user. In an embodiment, an input device 114, including alphanumeric and other keys, is coupled to bus 102 for communicating information and command selections to processor 104. Another type of user input device is cursor control 116, such as a mouse, a trackball, or cursor direction keys for communicating direction information and command selections to processor 104 and for controlling cursor movement on display 112. This input device typically has two degrees of freedom in two axes, a first axis (e.g., x) and a second axis (e.g., y), that allows the device to specify positions in a plane. In an embodiment, a touch panel (screen) display may also be used as an input device. In an embodiment, an audio recorder may be used as an input device to receive audio signals, e.g., from a user.

According to an embodiment, portions of a method described herein may be performed by computer system 100 in response to processor 104 executing one or more sequences of one or more instructions contained in main memory 106. Such instructions may be read into main memory 106 from another computer-readable medium, such as storage device 110. Execution of the sequences of instructions contained in main memory 106 causes processor 104 to perform the process steps described herein. One or more processors in a multi-processing arrangement may also be employed to execute the sequences of instructions contained in main memory 106. In an embodiment, hard-wired circuitry may be used in place of or in combination with software instructions. Thus, the description herein is not limited to any specific combination of hardware circuitry and software.

Various forms of computer readable media may be involved in carrying one or more sequences of one or more instructions to processor 104 for execution. For example, the instructions may initially be borne on a magnetic disk of a remote computer. The remote computer can load the instructions into its dynamic memory and send the instructions over a communications line, such as a telephone line using a modem. The computer system 100 can receive the data on the line and to convert the data to a signal, Bus 102 can receive the data carried in the signal and carry the data to main memory 106, from which processor 104 retrieves and executes the instructions. The instructions received by main memory 106 may optionally be stored on storage device 110 either before or after execution by processor 104.

In an embodiment, computer system 100 includes a communication interface 118 coupled to bus 102. Communication interface 118 provides a two-way data communication coupling to a network link 120 that is connected to a local network 122. For example, communication interface 118 may be an integrated services digital network (ISDN) card or a modem to provide a data communication connection to a corresponding type of telephone line. As another example, communication interface 118 may be a local area network (LAN) card to provide a data communication connection to a compatible LAN. Wireless links may also be implemented. In any such implementation, communication interface 118 sends and receives electrical, electromagnetic or optical signals that carry digital data streams representing various types of information.

Network link 120 typically provides data communication through one or more networks to other data devices. For example, network link 120 may provide a connection through local network 122 to a host computer 124 or to data equipment operated by an Internet Service Provider (ISP) 126. ISP 126 in turn provides data communication services through the worldwide packet data communication network, now commonly referred to as the “Internet” 128. Local network 122 and Internet 128 both use electrical, electromagnetic or optical signals that carry digital data streams. The signals through the various networks and the signals on network link 120 and through communication interface 118, which carry the digital data to and from computer system 100, are example forms of carrier waves transporting the information.

Computer system 100 can send messages and receive data, including program code, through the network(s), network link 120, and communication interface 118. In the Internet example, a server 130 might transmit a requested code for an application program through Internet 128, ISP 126, local network 122 and communication interface 118. One such downloaded application may provide for the illumination optimization of the embodiment, for example. The received code may be executed by processor 104 as it is received, and/or stored in storage device 110, or other non-volatile storage for later execution. In this manner, computer system 100 may obtain application code in the form of a carrier wave.

It will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Embodiments of the disclosure may be implemented in hardware, firmware, software, or any combination thereof. Embodiments of the disclosure may also be implemented as instructions stored on a computer-readable medium, which may be read and executed by one or more processors. A computer-readable medium may include any mechanism that stores or transmits information in a form readable by a machine (e.g., a computing device). A computer-readable medium may be any medium that participates in providing instructions to processor 104 for execution. Any combination of one or more computer readable media may be utilized.

The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), any memory chip or cartridge, an appropriate optical fiber with a repeater, coaxial cables, copper wire, fiber optics, a portable compact disc read-only memory (CDROM), an optical storage device, a magnetic storage device, or any suitable combination of the foregoing. In the context of this document, a computer readable storage medium may be any tangible or non-transitory medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, acoustic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

Firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

The flowchart and block diagrams in the Figures illustrate the architecture, functionality, and operation of possible implementations of systems, methods and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of any means or step plus function elements in the claims below are intended to include any disclosed structure, material, or act for performing the function in combination with other claimed elements as specifically claimed. The description of the present disclosure has been presented for purposes of illustration and description, but is not intended to be exhaustive or limited to the disclosure in the form disclosed, Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

Other implementations, uses, and advantages of the disclosed technology will be apparent to those skilled in the art from consideration of the specification and practice of the disclosure herein. The specification should be considered exemplary only, and the scope of the technology disclosed herein is accordingly intended to be limited only by any associated claims.

1. A method for a physical product manufacturing process, the method comprising:
identifying a critical quality attribute (CQA) of a physical product manufactured by the process;
identifying an acceptance criterion for the CQA for single and/or multiple units of the product manufactured by the process; and
based on data collected for the CQA of a plurality of units of the product manufactured by the process, determining, by a hardware computer processor, a Probability of Acceptance (Pa) value that specifies the probability that the acceptance criterion for a future unit of the product will meet or cross, or will not meet or not cross, a threshold defined for the acceptance criterion.

2. The method of claim 1, further comprising modifying the product manufacturing process based on the Probability of Acceptance (Pa) value.

3. The method of claim 2, further comprising modifying physical equipment or a physical test method.

4. (canceled)

5. The method of claim 1, further comprising manufacturing additional units of the product using the product manufacturing process provided the Probability of Acceptance (Pa) value meets or crosses a threshold.

6. The method of claim 1, further comprising designating units of the product manufactured by the product manufacturing process for sale or consumption provided the Probability of Acceptance (Pa) value meets or crosses a threshold.

7. The method of claim 1, further comprising determining an expected range for the acceptance criterion for the CQA.

8. The method of claim 1, wherein the acceptance criterion is a single-stage acceptance criterion for the CQA.

9.-10. (canceled)

11. The method of claim 8, wherein a single-stage acceptance criterion is applied to multiple test results.

12. (canceled)

13. The method of claim 11, wherein the Probability of Acceptance (Pa) value is, or is derived from, (Pₛᵤ)ⁿ wherein: n=number of units tested and Pₛᵤ=probability that a single unit will pass an acceptance criteria.

14. The method of claim 1, wherein the acceptance criterion comprises, or is derived using, multi-stage acceptance criteria.

15. The method of claim 14, wherein the multi-stage acceptance criterion is applied to a single test result.

16. (canceled)

17. The method of claim 14, wherein the multi-stage acceptance criterion is applied to multiple test results.

18. (canceled)

19. The method of claim 14, wherein the CQA comprises content uniformity.

20. The method of claim 14, wherein the expected range for the acceptance criterion is determined using:
$${S}_{lo} = s \sqrt{\frac{n - 1}{{X}_{( n - 1 , 1 - \frac{a}{2} )}^{2}}}$$
$${s}_{h i} = s \sqrt{\frac{n - 1}{{X}_{( n - 1 , \frac{a}{2} )}^{2}}} .$$
wherein: n=the number of units tested, s=sample standard deviation, sₗₒ=lower 95% confidence level of the standard deviation, sₕᵢ=upper 95% confidence level of the standard deviation, χ²=chi-squared distribution, and α=probability that units of the product exceed an acceptance value limit.

21. The method of claim 14, wherein the Probability of Acceptance value (Pa) is, or is derived from, α, wherein α is calculated from:
$${X}_{( n - 1 , \frac{a}{2} )}^{2} = \frac{( n - 1 ) {s}^{2}}{{s}_{l im}^{2}}$$
wherein n=the number of units of the product tested, s=measured standard deviation, sₗᵢₘ=limiting standard deviation, and χ²=chi-squared distribution.

22. The method of claim 14, further comprising running a statistical simulation of the product manufacturing process and determining a Probability of Acceptance (Pa) value for a CQA with the multi-stage acceptance criterion based on data from the simulation.

23. (canceled)

24. The method of claim 1, further comprising determining whether the data collected has a normal or non-normal distribution, and determining the Probability of Acceptance (Pa) value using data that has a normal distribution.

25.-34. (canceled)

35. The method of claim 1, wherein the Probability of Acceptance (Pa) value is used to determine the need for additional testing.

36. A method for a physical product manufacturing process, the method comprising:
identifying a critical quality attribute (CQA) of a physical product manufactured by the process;
identifying a plurality of acceptance criteria for the CQA, wherein a first acceptance criterion of the plurality of acceptance criteria is dependent on a second acceptance criterion of the plurality of acceptance criteria; and
based on at least the first and second acceptance criteria and on data collected for the CQA of a plurality of units of the product manufactured by the process, determining, by a hardware computer processor, a Probability of Acceptance (Pa) value that specifies the probability that the first acceptance criterion and/or the second acceptance criterion for a future unit of the product will meet or cross, or will not meet or not cross, a threshold defined for the respective first criterion and/or second criterion.

37. A computer program product comprising a non-transitory computer readable storage medium comprising computer-readable program code embodied therewith, the computer readable program code, when executed by a computer system, configured to cause the computer system to at least:
identify a critical quality attribute (CQA) of a physical product manufactured by a physical manufacturing process;
identify an acceptance criterion for the CQA for single and/or multiple units of the product manufactured by the process; and
based on data collected for the CQA of a plurality of units of the product manufactured by the process, determine a Probability of Acceptance (Pa) value that specifies the probability that the acceptance criterion for a future unit of the product will meet or cross, or will not meet or not cross, a threshold defined for the acceptance criterion.

38.-44. (canceled)
